(19) United States
(12) Patent Application Publication
(10) Pub. No.: US 2024/0108595 A1
(43) Pub. Date: Apr. 4, 2024

(54) COMPOSITIONS AND METHODS FOR REVERSING AGE-ASSOCIATED DENDRITIC SPINE LOSS

(71) Applicants: UNIVERSITY OF PITTSBURGH - OF THE COMMONWEALTH SYSTEM OF HIGHER EDUCATION, Pittsburgh, PA (US); UNIVERSITY OF PITTSBURGH MEDICAL CENTER, Pittsburgh, PA (US)

(72) Inventors: Robert A. SWEET, Pittsburgh, PA (US); Brandon C. MCKINNEY, Pittsburgh, PA (US); LiRong WANG, Pittsburgh, PA (US); Matthew Luke MACDONALD, Pittsburgh, PA (US); Peihao FAN, Pittsburgh, PA (US); Joshua Michael KRIVINKO, Pittsburgh, PA (US)

(21) Appl. No.: 18/464,725

(22) Filed: Sep. 11, 2023

Related U.S. Application Data
(60) Provisional application No. 63/404,994, filed on Sep. 9, 2022.

(51) Int. Cl. A61K 31/221; A61K 31/42; A61K 31/436; A61K 31/497; A61K 31/506; A61K 31/519; A61K 31/5685; A61K 31/737; A61P 25/28

(52) U.S. Cl. CPC A61K 31/221; A61K 31/42; A61K 31/436; A61K 31/497; A61K 31/506; A61K 31/519; A61K 31/5685; A61K 31/737; A61P 25/28

(57) ABSTRACT

Described herein are methods of treating, preventing, or reducing dendritic spine density loss in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or compositions described herein.

### CROSS-REFERENCE TO RELATED APPLICATION

This application claims priority to U.S. Provisional Application 63/404,994, filed on Sep. 9, 2022, the contents of which is hereby incorporated in its entirety.

### STATEMENT REGARDING FEDERALLY SPONSORED RESEARCH OR DEVELOPMENT

This invention was made with government support under Grant No. MH116046 awarded by National Institutes of Health. The government has certain rights in the invention.

### REFERENCE TO LARGE TABLES

Large Table submitted Sep. 11, 2023 as a text filed named “Table5.txt” created Sep. 11, 2023, and having a file size of 214,011 bytes is hereby incorporated by reference pursuant to 37 C.F.R. § 1.52(e)(8).

Large Table submitted Sep. 11, 2023 as a text filed named “Table6.txt” created Sep. 11, 2023, and having a file size of 204,695 bytes is hereby incorporated by reference pursuant to 37 C.F.R. § 1.52(e)(8).

Large Table submitted Sep. 11, 2023 as a text filed named “Table7.txt” created Sep. 11, 2023, and having a file size of 230,846 bytes is hereby incorporated by reference pursuant to 37 C.F.R. § 1.52(e)(8).

### BACKGROUND

Throughout adult life, the number of dendritic spines, a component of excitatory synapses, decline in the cerebral cortex. Dendritic spines, specialized receptive contacts which protrude from the dendritic shaft, are the morphologic correlate of the excitatory post-synapse. Disease-focused reports have documented that the density of dendritic spines in select brain areas is an important correlate of cognitive decline in several neurocognitive disorders including the most common neurocognitive disorder, Alzheimer Disease (AD).

Accordingly, there is a need for compounds and compositions useful to treat, prevent, or reduce age associated dendritic spine density loss.

The compositions and methods disclosed herein address these and other needs.

### SUMMARY

Provided herein are methods of treating, preventing, or reducing dendritic spine density loss in a subject. The method can include administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, the methods described herein can increase or decrease the activity of a mediator protein selected from the list of mediator proteins in Tables 3A-3E in a subject. In some embodiments, the method increases the activity of a mediator protein selected from the list of mediator protein in Tables 3B and 3D in a subject. In some embodiments, the methods decrease the activity of a mediator protein selected from the list of mediator proteins in Tables 3C and 3E in a subject. In some embodiments, described herein are also methods of enhancing cognitive resilience in a subject. In some embodiments, the methods described herein can protect against neurodegenerative dementia onset.

In some embodiments, the subject can have age related cognitive decline, age-associated dendritic spine density loss, or any combination thereof. In some embodiments, the subject can have dementia. In some embodiments, the dementia can be associated with a condition including, but not limited to, Alzheimer's disease, Lewy-Body disease, Parkinson's disease, frontotemporal dementia, vascular dementia, mixed dementia, progressive dementia, Huntington's disease, cortical dementia, subcortical dementia, corticobasal degeneration, progressive supranuclear palsy, limbic-predominant age-related TDP-43 encephalopathy (LATE), traumatic brain injury, Creutzfeldt-Jakob disease, Wernicke-korsakoff syndrome, normal pressure hydrocephalus, multiple sclerosis, HIV-associated dementia, chronic traumatic encephalopathy, argyrophilic grain disease, autism spectrum disorders (ASDs), and schizophrenia.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1C. Correlations of age and density of small, medium, and large dendritic spines within the precuneus. The age and density of small (1A), medium (1B), and large (1C) dendritic spines within the precuneus for each subject is shown. The dashed lines denote linear regression lines. Only the correlation of age and density of large dendritic spines (1C) is significant (p=<0.01). Small dendritic spines are those <0.45 um³, medium dendritic spines are 0.45-0.90 um³, and large dendritic spines are >0.90 um³.

FIG. 2A-2B. Correlations of age and protein abundance within homogenate and synaptosome fractions. The regression coefficients and regression p values on a -logio scale for each protein in homogenate (2A) and synaptosome (2B) fractions is shown. The horizontal dashed line denotes the false discovery threshold q value<0.05); points in gray below the dashed lines denote proteins whose abundance levels are not significantly correlated with age, while points above the dashed line denote proteins whose abundance levels are significantly correlated with age.

FIG. 3A-3D. Identification of the precuneus area. A sagittal schematic (3A) and medial view of flash frozen tissue from a subject (3B) are shown with annotated boarders used to identify the precuneus. The precuneus (Pc) was identified using the marginal branch of the cingulate sulcus (MBCS) as the anterior boarder (blue), the subparietal sulcus (SPS) as the inferior boarder (red), and the parietooccipital sulcus (POS) as the posterior boarder (gray). The entire block of isolated precuneus tissue (3C) along with a mounted block of precuneus tissue containing microdissection score marks for white and gray matter separation (3D) from the same subject are shown.

FIG. 4A-4J. Representative images from a 52-year-old subject demonstrating methodologic approach to quantifying dendritic spine density. Representative images from a male aged 52 years depicting spinophilin (green), phalloidin (red), and merged (yellow) immunofluorescence channels before (4A-4C) and after blind deconvolution (4D-4F) and Gaussian subtraction (4J-4I). Scale bars of 10 μm length are contained in the lower left corner of each image. Each dendritic spine was binned according to its size into small (<0.45 um³), medium (0.45-0.90 um³), or large (>0.90 um³) categories (4J).

FIG. 5A-5K. Correlations of age and dendritic spine density across dendritic spine size categories (Bins). The age and dendritic spine density across 10 dendritic spine size categories within the precuneus for each subject is shown (5A-5J). The dashed lines denote linear regression lines. Bonferroni-corrected p values for the correlations of age and dendritic spine density for each size bin are shown. A summary of Pearson R correlation values for each dendritic spine size category is shown (5K). Dendritic spine size categories 1-3, 4-6, and 7-10 were collapsed into small, medium, and large size as noted in (5K).

FIG. 6. Schematic depicting statistical mediation analysis. The outcome variable is the density of large dendritic spines. The product of the effect of age on protein abundance (a) and the effect of the protein on dendritic spine density (b) is the mediation effect. The direct effect of age on dendritic spine density (c) is the effect of age after controlling for the effect of the protein. The sum of the mediation effect and the direct age effect is the total effect of age on dendritic spine density.

FIG. 7 shows a graph of relative abundance (Amlexanox/Control) of each protein on a log2 scale.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

An “increase” can refer to any change that results in a larger amount of activity or level. A substance is also understood to increase the genetic output of a gene when the genetic output of the gene product with the substance is more relative to the output of the gene product without the substance. Also, for example, an increase can be a change in the symptoms of a disorder such that the symptoms are less than previously observed. An increase can be any individual, median, or average increase in an activity or level in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

### Composition

Described herein are pharmaceutical compositions including an effective amount of metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; and a pharmaceutically acceptable carrier.

### Metformin

Metformin is a compound of formula I:

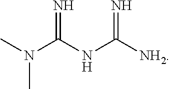

Metformin is a biguanide class of drug. It is marketed as immediate release formulations in the form of its hydrochloride salt (such as Glucophage CO and so-called extended release formulations (Fortamet®, Glucophage XR®, and Glumetza®). As referenced here metformin or a pharmaceutically acceptable salt, prodrug, or derivative thereof are disclosed generically, sub generically and specifically in any one or more of International Patent Publication Nos. WO2010/100337, and WO2004/004774; U.S. Patent Publication Nos. US2011/0257432; U.S. Pat. Nos. 8,853,259, 9,150,504, 9,815,777, 9,862,693, 10,017,529, 10,988,471 (Table 4), and 11,065,215, each of which is incorporated herein by reference in its entirety.

### Pentosan Polysulfate

Pentosan polysulfate is a compound of formula II:

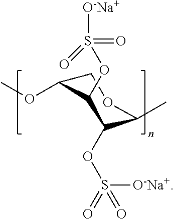

As referenced here sulfated xylans such as pentosan polysulfate or a pharmaceutically acceptable salt, prodrug, or derivative thereof are disclosed generically, sub generically and specifically in U.S. Patent Publication No. US20210128606, which is incorporated herein by reference in its entirety.

### Amlexanox

Amlexanox is a compound of formula III:

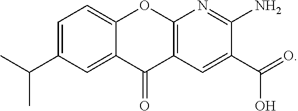 [SMILES: CC(C)c1ccc2oc3nc(N)c(C(=O)O)cc3c(=O)c2c1]

As referenced here amlexanox or a pharmaceutically acceptable salt, prodrug, or derivative thereof are disclosed generically, sub generically and specifically in any one or more of U.S. Patent Publication Nos. US2018/0230161; and U.S. Pat. No. 4,143,042, each of which is incorporated herein by reference in its entirety.

### Leflunomide

Leflunomide is a compound of formula IV:

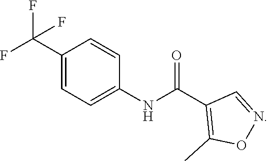 [SMILES: Cc1oncc1C(=O)Nc1ccc(C(F)(F)F)cc1]

Leflunomide is the prodrug of teriflunomide sold under the trade name Arava®.

As referenced here leflunomide or a pharmaceutically acceptable salt, prodrug, or derivative thereof are disclosed generically, sub generically and specifically in any one or more of International Patent Publication Nos. WO 97/34600, WO2002072527; and U.S. Pat. No. 7,071,222.

### Prasterone

Prasterone is a compound of formula V:

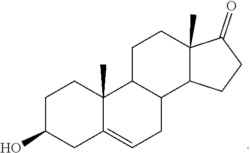 [SMILES: C[C@]12CC[C@H](O)CC1=CCC1C2CC[C@]2(C)C(=O)CCC12]

Prasterone also known as dehydroepiandrosterone (DHEA) is sold under the brand names Intrarosa®, Diandrone®, and Gynodian Depot® among others.

As referenced here prasterone or a pharmaceutically acceptable salt, prodrug, or derivative thereof are disclosed generically, sub generically and specifically in any one or more of International Patent Publication No. WO 91/04030, WO2008110941; U.S. Pat. Nos. 4,956,355, 4,898,694, 5,001,119, 5,028,631, 5,175,154, 5,527,789, 5,922,701, 6,087,351, and 7,405,207.

### Glutathione Disulfide

Glutathione disulfide is a compound of formula VI:

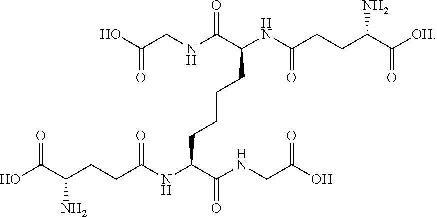 [SMILES: N[C@@H](CCC(=O)N[C@@H](CCCC[C@H](NC(=O)CC[C@H](N)C(=O)O)C(=O)NCC(=O)O)C(=O)NCC(=O)O)C(=O)O]

### Pazopanib

Pazopanib is a compound of formula VII:

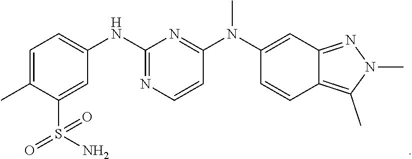

Pazopanib is a tyrosine kinase indicator, belonging to a class of substituted pyrimidine derivative. Pazopanib is marketed in a hydrochloride salt form and is sold under the trade name VOTRIENT®.

As referenced here pazopanib or a pharmaceutically acceptable salt, prodrug, or derivative thereof are disclosed generically, sub generically and specifically in any one or more of U.S. Pat. Nos. 7,105,530, 7,262,203, and 8,114,885.

### Bosutinib

Bosutinib is a compound of formula VIII:

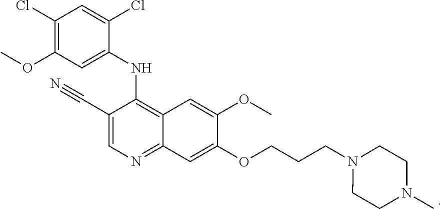

Bosutinib is a kinase inhibitor and is the active pharmaceutical ingredient in the BOSULIF®.

As referenced here bosutinib or a pharmaceutically acceptable salt, prodrug, or derivative thereof are disclosed generically, sub generically and specifically in any one or more of U.S. Pat. Nos. 6,002,008, and 9,776,970.

### Dabrafenib

Dabrafenib is a compound of formula IX:

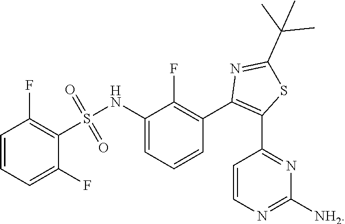 [SMILES: CC(C)(C)c1nc(-c2cccc(NS(=O)(=O)c3c(F)cccc3F)c2F)c(-c2ccnc(N)n2)s1]

Dabrafenib is a benzene sulfonamide thiazole compound and is a selective BRAF kinase inhibitor. Dabrafenib is preferably administered as its mesylate salt

As referenced here dabrafenib or a pharmaceutically acceptable salt, prodrug, or derivative thereof are disclosed generically, sub generically and specifically in any one or more of U.S. Pat. Nos. 7,994,185, 8,415,345, 9,233,956, 8,703,781, 10,869,869, and 9,453,011.

### Baricitinib

Baricitinib is a compound of formula X:

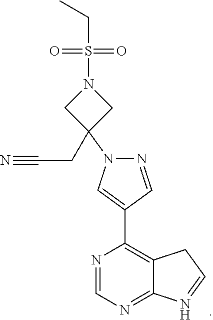 [SMILES: CCS(=O)(=O)N1CC(CC#N)(n2cc(-c3ncnc4c3CC=N4)cn2)C1]

Barcitinib is a pyrrolopyrimidine and a selective Janus Kinase 1 and 2 (JAK1 and JAK2) inhibitor. Baricitinib is sold under the brand name Olumiant®.

As referenced here barcitinib or a pharmaceutically acceptable salt, prodrug, or derivative thereof are disclosed generically, sub generically and specifically in any one or more of U.S. Pat. Nos. 8,420,629, 8,158,616, and 10,766,900.

In some embodiments, the pharmaceutical compositions can include metformin, pentosan polysulfate, amlexanox, leflunomide, prasterone, glutathione disulfide, pazopanib, bosutinib, dabrafenib, or baricitinib, or a pharmaceutically acceptable salt, prodrug, or derivative thereof. In some embodiments, the pharmaceutical compositions can include metformin or a pharmaceutically acceptable salt, prodrug, or derivative thereof. In some embodiments, the pharmaceutical compositions can include pentosan polysulfate or a pharmaceutically acceptable salt, prodrug, or derivative thereof. In some embodiments, the pharmaceutical compositions can include amlexanox or a pharmaceutically acceptable salt, prodrug, or derivative thereof. In some embodiments, the pharmaceutical compositions can include leflunomide or a pharmaceutically acceptable salt, prodrug, or derivative thereof. In some embodiments, the pharmaceutical compositions can include prasterone or a pharmaceutically acceptable salt, prodrug, or derivative thereof. In some embodiments, the pharmaceutical compositions can include glutathione disulfide or a pharmaceutically acceptable salt, prodrug, or derivative thereof. In some embodiments, the pharmaceutical compositions can include pazopanib or a pharmaceutically acceptable salt, prodrug, or derivative thereof. In some embodiments, the pharmaceutical compositions can include bosutinib or a pharmaceutically acceptable salt, prodrug, or derivative thereof. In some embodiments, the pharmaceutical compositions can include dabrafenib or a pharmaceutically acceptable salt, prodrug, or derivative thereof. In some embodiments, the pharmaceutical compositions can include baricitinib or a pharmaceutically acceptable salt, prodrug, or derivative thereof.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to treat age-associated dendritic spine density loss in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to prevent age-associated dendritic spine density loss in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to reduce age-associated dendritic spine density loss in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to prevent dementia onset in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to enhance cognitive resilience in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to prevent age related cognitive decline in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to treat age related cognitive decline in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to increase the activity of a mediator protein selected from the list of mediator proteins in Tables 3B and 3D in a subject.

In some embodiments, glutathione disulfide, prasterone, or a pharmaceutically acceptable salt, prodrug, or derivative thereof; or any combination thereof can be present in the composition in an effective amount to increase the activity of a mediator protein selected from the list of mediator proteins in Tables 3B and 3D in a subject.

In some embodiments, metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof can be present in the composition in an effective amount to decrease the activity of a mediator protein selected from the list of mediator proteins in Tables 3C and 3E in a subject.

In some embodiments, metformin, pentosan polysulfate, amlexanox, leflunomide, pazopanib, bosutinib, dabrafenib, baricitinib, or a pharmaceutically acceptable salt, prodrug, or derivative thereof; or any combination thereof can be present in the composition in an effective amount to decrease the activity of a mediator protein selected from the list of mediator proteins in Tables 3C and 3E in a subject.

The term “pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylc arboxymethylcellulo se (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard- filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of the active agent or a pharmaceutically acceptable salt or derivative thereof.

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. The active agent may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc. Administration of the active components of their compositions can be a single administration, or at continuous and distinct intervals as can be readily determined by a person skilled in the art.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Methods of Use

Described herein are methods of treating, preventing, or reducing age-associated dendritic spine density loss in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, described are methods of treating age-associated dendritic spine density loss in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, described are methods of preventing age-associated dendritic spine density loss in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, described are methods of reducing age-associated dendritic spine density loss in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, the methods described herein can protect against neurodegenerative dementia onset. In some embodiments, the methods described herein can prevent or reduce age related cognitive decline.

Described herein are methods of enhancing cognitive resilience comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, the compound can be selected from metformin, pentosan polysulfate, amlexanox, leflunomide, prasterone, glutathione disulfide, pazopanib, bosutinib, dabrafenib, or baricitinib or a pharmaceutically acceptable salt, prodrug, or derivative thereof. In some embodiments, the compound can be metformin or a pharmaceutically acceptable salt, prodrug, or derivative thereof. In some embodiments, the compound can be pentosan polysulfate or a pharmaceutically acceptable salt, prodrug, or derivative thereof. In some embodiments, the compound can be amlexanox or a pharmaceutically acceptable salt, prodrug, or derivative thereof. In some embodiments, the compound can be leflunomide or a pharmaceutically acceptable salt, prodrug, or derivative thereof. In some embodiments, the compound can be prasterone or a pharmaceutically acceptable salt, prodrug, or derivative thereof. In some embodiments, the compound can be glutathione disulfide or a pharmaceutically acceptable salt, prodrug, or derivative thereof. In some embodiments, the compound can be pazopanib or a pharmaceutically acceptable salt, prodrug, or derivative thereof. In some embodiments, the compound can be bosutinib or a pharmaceutically acceptable salt, prodrug, or derivative thereof. In some embodiments, the compound can be dabrafenib or a pharmaceutically acceptable salt, prodrug, or derivative thereof. In some embodiments, the compound can be baricitinib or a pharmaceutically acceptable salt, prodrug, or derivative thereof. In some embodiments, the compound can be metformin, pentosan polysulfate, amlexanox, leflunomide, prasterone, glutathione disulfide, pazopanib, bosutinib, dabrafenib, and baricitinib or a pharmaceutically acceptable salt, prodrug, or derivative thereof. In some embodiments, the compound can be selected from: glutathione disulfide, prasterone, or a pharmaceutically acceptable salt, prodrug, or derivative thereof; or any combination thereof. In these embodiments, the compound can be selected from: metformin, pentosan polysulfate, amlexanox, leflunomide, pazopanib, bosutinib, dabrafenib, or baricitinib, or a pharmaceutically acceptable salt, prodrug, or derivative thereof.

In some embodiments, the compound can be metformin and pentosan polysulfate, or a pharmaceutically acceptable salt, prodrug, or derivative thereof. In some embodiments, the compound can be metformin and amlexanox, or a pharmaceutically acceptable salt, prodrug, or derivative thereof. In some embodiments, the compound can be metformin and leflunomide, or a pharmaceutically acceptable salt, prodrug, or derivative thereof. In some embodiments, the compound can be metformin and prasterone, or a pharmaceutically acceptable salt, prodrug, or derivative thereof. In some embodiments, the compound can be metformin and glutathione disulfide, or a pharmaceutically acceptable salt, prodrug, or derivative thereof. In some embodiments, the compound can be metformin and pazopanib, or a pharmaceutically acceptable salt, prodrug, or derivative thereof. In some embodiments, the compound can be metformin and bosutinib, or a pharmaceutically acceptable salt, prodrug, or derivative thereof. In some embodiments, the compound can be metformin and dabrafenib, or a pharmaceutically acceptable salt, prodrug, or derivative thereof. In some embodiments, the compound can be metformin and baricitinib, or a pharmaceutically acceptable salt, prodrug, or derivative thereof.

In some embodiments, the compound can be pentosan polysulfate and amlexanox, or a pharmaceutically acceptable salt, prodrug, or derivative thereof. In some embodiments, the compound can be pentosan polysulfate and leflunomide, or a pharmaceutically acceptable salt, prodrug, or derivative thereof. In some embodiments, the compound can be pentosan polysulfate and prasterone, or a pharmaceutically acceptable salt, prodrug, or derivative thereof. In some embodiments, the compound can be pentosan polysulfate and glutathione disulfide, or a pharmaceutically acceptable salt, prodrug, or derivative thereof. In some embodiments, the compound can be pentosan polysulfate and pazopanib, or a pharmaceutically acceptable salt, prodrug, or derivative thereof. In some embodiments, the compound can be pentosan polysulfate and bosutinib, or a pharmaceutically acceptable salt, prodrug, or derivative thereof. In some embodiments, the compound can be pentosan polysulfate and dabrafenib, or a pharmaceutically acceptable salt, prodrug, or derivative thereof. In some embodiments, the compound can be pentosan polysulfate and baricitinib, or a pharmaceutically acceptable salt, prodrug, or derivative thereof.

In some embodiments, the compound can be amlexanox and leflunomide, or a pharmaceutically acceptable salt, prodrug, or derivative thereof. In some embodiments, the compound can be amlexanox and prasterone, or a pharmaceutically acceptable salt, prodrug, or derivative thereof. In some embodiments, the compound can be amlexanox and glutathione disulfide, or a pharmaceutically acceptable salt, prodrug, or derivative thereof. In some embodiments, the compound can be amlexanox and pazopanib, or a pharmaceutically acceptable salt, prodrug, or derivative thereof. In some embodiments, the compound can be amlexanox and bosutinib, or a pharmaceutically acceptable salt, prodrug, or derivative thereof. In some embodiments, the compound can be amlexanox and dabrafenib, or a pharmaceutically acceptable salt, prodrug, or derivative thereof. In some embodiments, the compound can be amlexanox and baricitinib, or a pharmaceutically acceptable salt, prodrug, or derivative thereof.

In some embodiments, the compound can be leflunomide and prasterone, or a pharmaceutically acceptable salt, prodrug, or derivative thereof. In some embodiments, the compound can be leflunomide and glutathione disulfide, or a pharmaceutically acceptable salt, prodrug, or derivative thereof. In some embodiments, the compound can be leflunomide and pazopanib, or a pharmaceutically acceptable salt, prodrug, or derivative thereof. In some embodiments, the compound can be leflunomide and bosutinib, or a pharmaceutically acceptable salt, prodrug, or derivative thereof. In some embodiments, the compound can be leflunomide and dabrafenib, or a pharmaceutically acceptable salt, prodrug, or derivative thereof. In some embodiments, the compound can be leflunomide and baricitinib, or a pharmaceutically acceptable salt, prodrug, or derivative thereof.

In some embodiments, the compound can be prasterone and glutathione disulfide, or a pharmaceutically acceptable salt, prodrug, or derivative thereof. In some embodiments, the compound can be prasterone and pazopanib, or a pharmaceutically acceptable salt, prodrug, or derivative thereof. In some embodiments, the compound can be prasterone and bosutinib, or a pharmaceutically acceptable salt, prodrug, or derivative thereof. In some embodiments, the compound can be prasterone and dabrafenib, or a pharmaceutically acceptable salt, prodrug, or derivative thereof. In some embodiments, the compound can be prasterone and baricitinib, or a pharmaceutically acceptable salt, prodrug, or derivative thereof.

In some embodiments, the compound can be glutathione disulfide and pazopanib, or a pharmaceutically acceptable salt, prodrug, or derivative thereof. In some embodiments, the compound can be glutathione disulfide and bosutinib, or a pharmaceutically acceptable salt, prodrug, or derivative thereof. In some embodiments, the compound can be glutathione disulfide and dabrafenib, or a pharmaceutically acceptable salt, prodrug, or derivative thereof. In some embodiments, the compound can be glutathione disulfide and baricitinib, or a pharmaceutically acceptable salt, prodrug, or derivative thereof. In some embodiments, the compound can be pazopanib and bosutinib, or a pharmaceutically acceptable salt, prodrug, or derivative thereof. In some embodiments, the compound can be pazopanib and dabrafenib, or a pharmaceutically acceptable salt, prodrug, or derivative thereof. In some embodiments, the compound can be pazopanib and baricitinib, or a pharmaceutically acceptable salt, prodrug, or derivative thereof. In some embodiments, the compound can be bosutinib and dabrafenib, or a pharmaceutically acceptable salt, prodrug, or derivative thereof. In some embodiments, the compound can be bosutinib and baricitinib, or a pharmaceutically acceptable salt, prodrug, or derivative thereof. In some embodiments, the compound can be dabrafenib and baricitinib, or a pharmaceutically acceptable salt, prodrug, or derivative thereof.

Described herein are methods of increasing or decreasing the activity of a mediator protein selected from the list of mediator proteins in Tables 3B and 3D or Tables 3C and 3E, the method comprising administering a compound selected from metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof; or a pharmaceutical composition described herein.

In some embodiments, described herein are methods of increasing the activity of a mediator protein selected from the list of mediator protein in Tables 3B and 3D in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof. In these embodiments, the compound can be selected from: glutathione disulfide, prasterone, or a pharmaceutically acceptable salt, prodrug, or derivative thereof; or any combination thereof.

In some embodiments, described herein are methods of decreasing the activity of a mediator protein selected from the list of mediator protein in Tables 3C and 3E in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof. In these embodiments, the compound can be selected from: metformin, pentosan polysulfate, amlexanox, leflunomide, pazopanib, bosutinib, dabrafenib, or baricitinib, or a pharmaceutically acceptable salt, prodrug, or derivative thereof.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human

In some embodiments, the subject can have age related cognitive decline. In some embodiments, the subject can have age-associated dendritic spine density loss. In some embodiments, the subject can have condition related to age-associated dendritic spine density loss. In some embodiments, the subject can have a condition related to age related cognitive decline. In some embodiments, the subject can have dementia. In some embodiments, the dementia can be associated with a condition including, but not limited to, Alzheimer's disease, Lewy-Body disease, Parkinson's disease, frontotemporal dementia, vascular dementia, mixed dementia, progressive dementia, Huntington's disease, cortical dementia, subcortical dementia, corticobasal degeneration, progressive supranuclear palsy, limbic-predominant age-related TDP-43 encephalopathy (LATE), traumatic brain injury, Creutzfeldt-Jakob disease, Wernicke-korsakoff syndrome, normal pressure hydrocephalus, multiple sclerosis, HIV-associated dementia, chronic traumatic encephalopathy, argyrophilic grain disease, autism spectrum disorders (ASDs), and schizophrenia. Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1

### Loss of Large Dendritic Spines During Normal Aging is Mediated by Alterations in Discrete Protein Levels Within The Precuneus

Dendritic spines, specialized receptive contacts which protrude from the dendritic shaft, are the morphologic correlate of the excitatory post-synapse (reviewed in [1]). Disease-focused reports have documented that the density of dendritic spines in select brain areas is an important correlate of cognitive decline in several neurocognitive disorders including the most common neurocognitive disorder, Alzheimer Disease (AD)[2]. For example, previous studies have documented reduced dendritic spine density[3] and synapse number[4] in AD subjects relative to control subjects, and preservation of dendritic spine density is considered a putative mechanism of cognitive resilience in asymptomatic subjects with AD-related neuropathology[5, 6, 35]. However, because normal aging is itself associated with dendritic spine loss (reviewed in [7]), studies designed a priori to identify upstream mediators of dendritic spine loss during normal aging may aid to develop targeted pharmacotherapies which enhance cognitive resilience to AD-related pathologies. Moreover, as normal aging is also associated with cognitive decline, treatments preventing or reversing dendritic spine loss during normal aging may also prevent or reverse normal age-related cognitive decline.

Prior reports have examined normative dendritic spine density alterations with chronological age[17]; however, such investigations have generally been restricted to a handful of brain areas, especially dorsolateral prefrontal cortex (DLPFC). Although DLPFC is a compelling candidate area for detecting markers of risk and resilience prior to AD onset[8], whether findings within DLPFC extend to other neocortical areas which are similarly susceptible to early AD-related changes remains unclear. The precuneus is an associative cortical area located within the posteriomedial cortex of the parietal lobe that is a highly functionally connected component of the default mode network, and involved in memory processing (Cavanna A E, et al. Brain. 2006; 129(Pt 3):564-83). Due to the advent of in vivo amyloid imaging, the precuneus is now appreciated as a selectively vulnerable region during AD pathogenesis, containing detectable amyloid-beta ligand binding in prodromal AD subjects and a linear increase in amyloid load [9] with concomitant reduction in metabolic activity[10, 11] during disease progression.

Therefore the effect of age on dendritic spine density was evaluated in the precuneus area using quantitative immunohistochemistry and confocal microscopy in a cohort of 98 subjects ages 20-96. Then quantitative liquid chromatography/mass spectrometry was conducted in precuneus gray matter homogenate and synaptosome fractions from the same individuals. A robust, negative correlation between chronological age and the density of large dendritic spines within the precuneus and uncovered was detected a significant correlation of age with 1839 of 5032 proteins detected in cellular homogenate and 914 of 4754 proteins detected in the synaptosome fraction. Which of the homogenate proteins statistically-mediated the effect of age on large dendritic spine loss was evaluated, identifying 203 protein mediators.

### Materials and Methods

### Sample Collection and Neuropathologic Assessment

In accordance with University of Pittsburgh's Committee for Oversight in Research Involving Decedents and the International Review Board for Biomedical Research, postmortem brain tissue was recovered from a total of 98 subjects during autopsies conducted at the Allegheny County Medical Examiner's Office in Pittsburgh, PA following acquisition of informed consent from next of kin. Consensus diagnoses, or absence thereof, were generated by an independent committee of experienced clinicians according to the Diagnostic and Statistical Manual of Mental Disorders, Fourth Edition, using data obtained from medical records, structured interviews with surviving relatives and, if available, toxicology reports. No subject included had a documented diagnosis of a neurocognitive disorder.

Subjects were evaluated by an experienced neuropathologist at time of autopsy with tissue sections from the frontal pole, hippocampus, entorhinal cortex, and cerebellum stained using hematoxylin and eosin, Bielschowsky silver stain, and amyloid β immunohistochemistry. No subject included was deemed to have sufficient evidence for a neuropathologic diagnosis of a neurodegenerative disease. The overall median age of the cohort was 51, with a minimum age of 20 years and a maximum age of 96 years. Subject demographics for each age group are contained in Table 1.

TABLE 1
Subject demographics by age group. For each age bracket, the number of subjects, mean age in
years, percent female sex, percent black race, mean [standard deviation] postmortem interval
(PMI) in hours, and mean [standard deviation] tissue storage time in months is shown.
The number of subjects found to have age-appropriate neuropathology is shown. A summary of neuropathologic
findings from all subjects with age-appropriate neuropathology is contained in Table 2. The
leading cause of death for each age bracket is also shown (percent of subjects affected).

| | | | | | | | Age-appropriate | |
|---|---|---|---|---|---|---|---|---|
| | | | | | | Mean storage | neuropathology*, | Leading cause |
| Age | | Mean age, | Sex, % | Race, % | Mean PMI, | time, months | number of subjects | of death (% of |
| Group | N | years | female | black | hours [SD] | [SD] | positive | subjects affected) |
| 20s | 16 | 25.1 | 31.3 | 37.5 | 15.0 | 151.5 | 0^{A} | Trauma (37.5) |
| | | | | | [6.6] | [76.3] | | |
| 30s | 16 | 36.0 | 43.8 | 31.3 | 16.7 | 137.9 | 1^{A} | Cardiovascular |
| | | | | | [7.1] | [67.0] | | disease (37.5) |
| 40s | 16 | 44.7 | 50.0 | 43.4 | 15.6 | 144.1 | 2^{B} | Cardiovascular |
| | | | | | [3.1] | [69.2] | | disease (62.5) |
| 50s | 16 | 55.3 | 43.8 | 43.4 | 15.2 | 137.8 | 1^{A} | Cardiovascular |
| | | | | | [3.3] | [69.6] | | disease (62.5) |
| 60s | 16 | 64.3 | 50.0 | 31.3 | 15.2 | 173.6 | 1^{C} | Cardiovascular |
| | | | | | [6.1] | [85.9] | | disease (43.8) |
| >70 | 18 | 77.6 | 38.9 | 11.1 | 18.8 | 167.8 | 7^{B} | Trauma (44.4) |
| | | | | | [5.6] | [62.9] | | |

*Age-appropriate neuropathology is defined as extracellular amyloid plaques, amyloid angiopathy, or neurofibrillary tangles present at levels not considered out of proportion for age according to an expert neuropathologist at time of autopsy.
Footnotes:
^{A}neuropathology results unavailable for 1 subject,
^{B}neuropathology results unavailable for 2 subjects,
^{C}neuropathology results unavailable for 4 subjects

TABLE 2
Results of neuropathology reports for all subjects containing age-
appropriate neuropathology. A summary of the neuropathologic findings for all 12
subjects found to have age-appropriate neuropathology (defined as the presence of
extracellular amyloid plaques, amyloid angiopathy, or neurofibrillary tangles) by an
experienced neuropathologist at time of autopsy is shown. No subject had
accumulation of neuropathologic species out of proportion for his/her age.

| | Age | Age | |
|---|---|---|---|
| Subject | group | (years) | Neuropathologic findings |
| 1 | 30 s | 38 | Rare neocortical diffuse amyloid plaques consistent with early |
| | | | mild age-related changes |
| 2 | 40 s | 47 | Mild Amyloid Angiopathy |
| 3 | 40 s | 47 | Moderate number of diffuse amyloid plaques, consistent with |
| | | | age |
| 4 | 50 s | 52 | Minimal amyloid plaque pathology, consistent with age |
| 5 | 60 s | 65 | Mild neurofibrillary tangle pathology, appropriate for age, |
| | | | corresponding to Braak Stage II/VI |
| 6 | >70 s | 73 | Mild neurofibrillary tangle pathology, appropriate for age, |
| | | | corresponding to Braak Stage II/VI |
| 7 | >70 s | 73 | Minimal neurofibrillary tangle pathology, appropriate for age, |
| | | | corresponding to Braak Stage I/VI |
| 8 | >70 s | 74 | Mild neurofibrillary tangle pathology, appropriate for age, |
| | | | corresponding to Braak stage II/VI |
| 9 | >70 s | 74 | Mild diffuse amyloid plaques, consistent with age |
| 10 | >70 s | 80 | Mild Amyloid Angiopathy |
| 11 | >70 s | 85 | Mild-moderate neurofibrillary tangle pathology, appropriate for |
| | | | age, corresponding to Braak Stage II-III/VI |
| 12 | >70 s | 96 | Moderate to frequent neocortical amyloid plaques and |
| | | | neurofibrillary tangles confined to the hippocampus and |
| | | | entorhinal cortex, appropriate for age |

### Tissue Collection

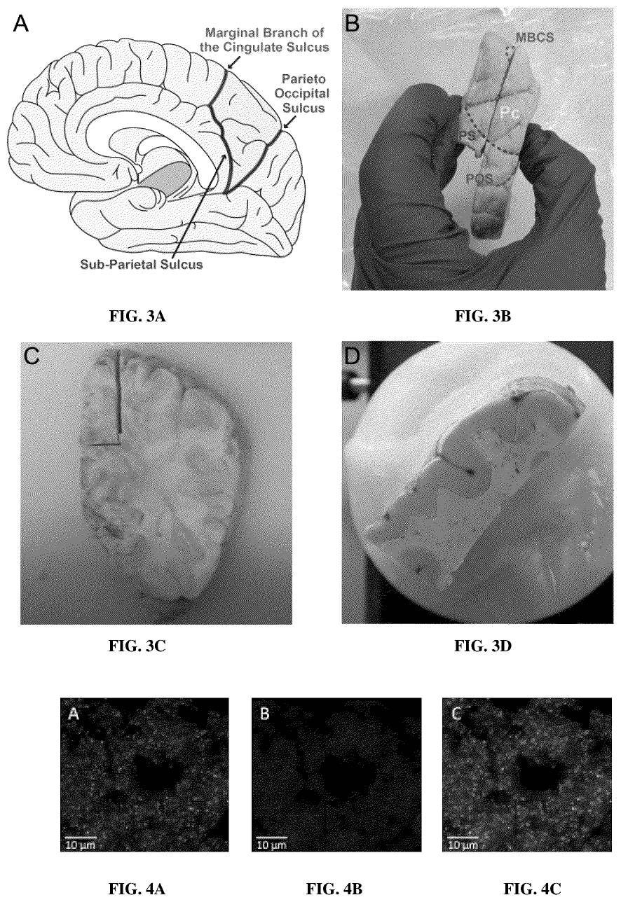

The precuneus from the right hemibrain was identified using the marginal branch of the cingulate sulcus as the anterior boarder, the subparietal sulcus as the inferior boarder, and the parietooccipital sulcus as the posterior boarder (FIG. 3A-3D). This area was isolated by dissection, and frozen at −80 for immunohistochemical and proteomic studies.

### Quantitative Immunohistochemistry

Two tissue sections containing the precuneus from each subject were obtained at a thickness of 20 μm, mounted onto Superfrost™ microscope slides (ThermoFisher Scientific, Waltham, MA), and stored at −80 C for immunohistochemistry (IHC). Subjects were assembled into eight blocks of 12-13 subjects per block. Two subjects from each age bracket (Table 1) were contained in each block of 12 subjects; for blocks of 13 subjects, three subjects from the >70 age group were included. IHC and microscopy assays were conducted in four runs with two blocks per run. Each subject was coded through IHC and microscopy assessments such that the experimenter (JN) was blind to subject age.

Slide-mounted tissue was fixed in cold 4% paraformaldehyde for 10 minutes, permeabilized for 30 minutes with 0.3% Triton X in phosphate buffered saline (PBS), and incubated in blocking buffer containing 20% normal goat serum (NGS, Jackson ImmunoResearch Laboratories, Inc, Westgrove, PA cat# 005-000-121), 20% normal human serum (NHS, Jackson ImmunoResearch Laboratories, Inc, Westgrove, PA, cat# 009-000-121), 1% bovine serum albumin (BSA), 0.1% glycine, and 0.1% lysine in PBS for 2.5 hours. Following removal of blocking buffer, sections were incubated in 5% NGS, 5% NHS, 1% BSA, 0.1% glycine, and 0.1% lysine in PBS with spinophilin antibody (dilution 1:1500, MilliporeSigma, Burlington, MA, cat# AB5669) for 24 hours at 4° C. Sections were then rinsed in PBS and incubated in darkness with Alexa Fluor 488 goat anti-rabbit secondary antibody (dilution 1:500, ThermoFisher Scientific, Waltham, MA, cat# A11034) and 3 units/μL Alexa Fluor 568 phalloidin (ThermoFisher Scientific, Waltham, MA, cat# A12380). Slides were then washed in PBS, cover slipped using VECTASHIELD® HardSetTM antifade mounting medium (Vector Laboratories, Burlingame, CA, cat# H-1400-10), and allowed to hard-set for at least 24 hours prior to handling and for an additional 24 hours at 4° C. before microscopy.

### Microscopy

Dendritic spines were visualized by colocalization of antibody-mediated detection of spinophilin and detection of filamentous actin by phalloidin as previously described1121. For each section, an area within the precuneus region was identified where the layer 6 white matter boundary was perpendicular to the pial surface and a total of 20 image sites were selected by a randomly rotating sample grid using Stereo Investigator Version 11 (MBF Bioscience, Williston, VT). At each site, 512×512 pixel sized 3D image stacks were captured at full measured tissue thickness using a 1.40 numerical aperture 60× oil super-corrected objective mounted on an Olympus BX51W1 upright microscope (Olympus, Center Valley, PA) equipped with an Olympus DSU spinning disk, Hamamatsu Orca R2 camera (Hamamatsu, Bridgewater, NJ), MBF CX9000 front-mounted digital camera (MicroBrightField Inc., Natick, MA), BioPrecision2 XYZ motorized stage with linear XYZ encoders (Ludl Electronic Products Ltd., Hawthorne, NY), excitation and emission filter wheels (Ludl Electronic Products Ltd., Hawthorne, NY), Sedat Quad 89000 filter set (Chroma Technology Corp., Bellows Falls, VT), and a Lumen 220 metal halide lamp (Prior Scientific, Rockland, MA). For 488 nm (spinophilin) and 568 nm (phalloidin) excitation wavelengths, exposure times were set to optimize the spread of the intensity histogram at each site. Lipofuscin was captured using the 405-excitation filter and 647-emission filter, though was not detected in the 568-channel with the exposure times used for capture, and therefore was not included in dendritic spine density calculations.

### Image Processing

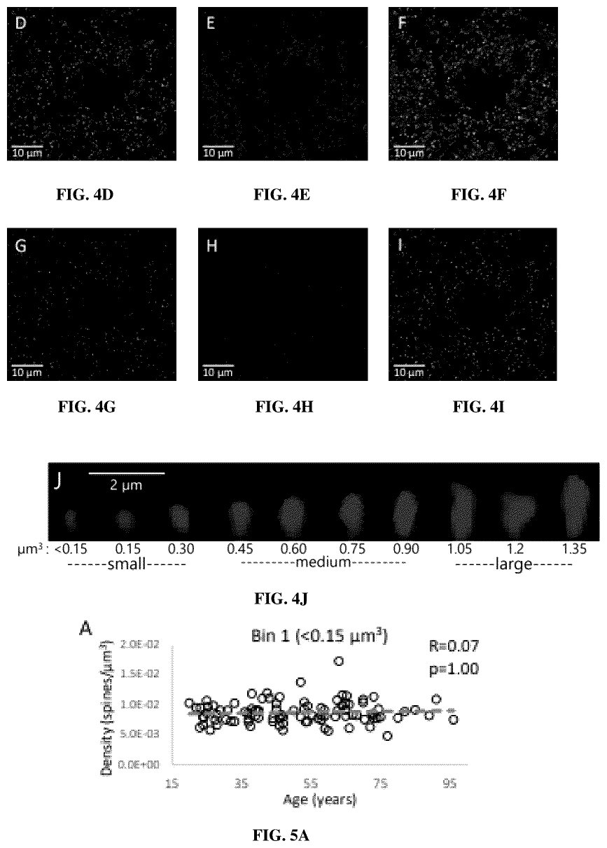

Image stacks were deconvolved with AutoQuant's blind deconvolution algorithm (MediaCybernetics, Rockville, MD) using Slidebook software version 6.015 (Intelligent Imaging Innovations, Inc, Denver, CO, version 6.015) and keystrokes automated by Automation Anywhere (Automation Anywhere, San Jose, CA, version 6). A gaussian channel was generated for phalloidin and spinophilin channels by calculating the difference of distributions using sigma values of 0.7 and 2.0 to define the edges of immunofluorescence puncta prior to data segmentation. FIG. 4 contains representative images from a male aged 52 years of spinophilin, phalloidin, and merged immunofluorescence channels before (4A-4C) and after blind deconvolution (4D-4F) and Gaussian subtraction (4G-4I). For each image stack, the intensity threshold for iterative segmentation for each channel was calculated using the Otsu iterative thresholding algorithm[13]. MATLAB (MathWorks, Inc., Natick, MA, version R2019b) was utilized to generate multiple iterations with subsequent threshold settings increasing by 25 gray levels. Object masks were size gated after each iteration as previously described[14] using ranges of 0.1-0.8 μm³ and 0.04-1.5 μm³ for the spinophilin and phalloidin channels, respectively, and together were then used to determine object intensities from devolved channels. Dendritic spines were defined as a ≥1 voxel overlap of a phalloidin masked object with a spinophilin masked object and categorized into one of 10 bins according to their volume as previously described[12, 14] to generate one dendritic spine density outcome per category per subject (FIG. 4J). For simplification, dendritic spine bins were subsequently aggregated into three size categories: small (bins 1-3, <0.45 um³), medium (bins 4-6, 0.45-0.90 um³), and large (bins 7-10, >0.90 um³).

### Tandem Mass Tag Mass Spectrometry

Subjects were coded to conceal subject age and sex throughout sample preparation for mass spectrometry. Samples were randomly assigned to one of eight TMT-plexes each containing 12-13 samples to be labeled with isobaric mass tags using a balanced block design such that age and sex was evenly distributed within each plex and across isobaric tags between plexes. Samples were retained in their respective plexes and plexes were randomly assigned to order of preparation at each step including homogenization, protein quantification, S-trap loading, isobaric tag labeling, and sample injection. Gray matter was dissected, weighed, and homogenized with a Dounce homogenizer on ice in 1 mL of Syn-Per reagent (Thermo Scientific #87793) with protease and phosphatase inhibitors. 100 μL of this homogenate was added to an equal volume of S-trap buffer containing 100 mM triethylammonium bicarbonate (TEAB) and 10% sodium dodecyl sulphate (SDS), to generate the total homogenate fraction, which was stored at −80° C. The remaining homogenate was centrifuged at 1200 g at 4° C. for 10 minutes, the pellet was discarded, and the supernatant centrifuged at 15000 g for 20 minutes at 4° C. The pellet was isolated and resuspended in 0.1 mM CaCl2 with protease and phosphatase inhibitors and samples were centrifuged at 15000 g for 20 minutes at 4° C., after which the supernatant was discarded and the pellet resuspended in S-trap buffer to generate the synaptosome fraction which was stored at −80° C.

Both homogenate and synaptosome fractions were then thawed at room temperature, bath sonicated for 10 minutes at 23° C., centrifuged at 13000 g for 10 minutes at room temperature, and the supernatants were isolated for total protein quantification by a micro-bicinchoninic acid assay (Thermo Scientific #23235). Samples were then subject to reduction with 20 mM dithiothreitol, heated to 95° C. for 10 mM, cooled to room temperature, and alkylated by incubation with 40 mM iodoacetamide in darkness for 30 min at room temperature. Samples were centrifuged at 13000 g for 8 mM, acidified with 12% phosphoric acid at 1:10 concentration, and diluted six-fold in binding buffer containing 90% methanol and 100 mM TEAB at pH 7.1. Samples were dispensed onto S-trap columns which were washed with binding buffer and centrifuged at 4000 g for 1 mM Columns were washed with twice concentrated binding buffer, centrifuged at 4000 g for 1 minute, and transferred to clean tubes for incubation at 47° C. in 50 mM TEAB for trypsin digestion at 1:10 enzyme/substrate. Samples were eluted with a series of solutions including 50 mM TEAB, 0.2% formic acid, and 50% acetonitrile with 0.2% formic acid, and underwent centrifugation at 1000 g for 1 mM after each eluent was added. Samples were then dried in a speedvac, resuspended in 100 mM TEAB, and labeled at a 1:4 ratio of peptide/TMTpro16 reagent. The pooled control was split into two equal aliquots and each was labeled separately with TMTpro16. Samples were pooled into plexes and each plex received aliquots of the two pooled control samples. Plexes were then fractionated using Pierce High pH Reversed-Phase Peptide Spin Columns (Thermo 84868). Peptides were eluted into 9 fractions using ACN at 5%, 10%, 12.5%, 15%, 17.5%, 20%, 22.5%, 25% and 50%. Samples were then dried in a speedvac and resuspended in a filtered solution of 3% acetonitrile and 0.1% formic acid to a final concentration of 0.5 μg/μL.

Data was collected using an Orbitrap Eclipse Tribrid Mass Spectrometer (ThermoFisher Scientific) coupled to an UltiMate 3000 UHPLC (ThermoFisher Scientific). Approximately 1 ug of each sample was injected onto an 50 cm EASY-Spray reversed phase column (ThermoFisher Scientific). Peptides were eluted over a 180-minute gradient optimized for high pH reversed-phase fractionation as previously described (Dumrongprechachan et al. 2021). MS1 spectra were collected at a resolution of 120,000 and MS2 were collected in the ion trap at with CID (35%). Ions for MS3 were selected using real time search (max search time=34 s; max missed cleavages=1; Xcorr=1; dCN=0.1; ppm=5) and MS3 spectra were collected in the Orbitrap with HCD (60%, isolation window=0.7 m/z; resolution=60,000; max injection time=400 ms).

Peptide identification and quantification was carried out in Proteome Discoverer (ThermoFisher Scientific). MS/MS spectra were searched against the human SwissProt database using Sequest HT (missed cleavages=2; minimum peptide length=6; precursor mass tolerance=10 ppm; dynamic modifications: Oxidation/+15.995 Da, Acetyl/+42.011 Da, Met-loss/−131.040 Da, Met-loss+Acetyl/−83.030 Da; static modifications: TMTpro/+304.207 Da, Carbamidomethyl/+57.021 Da). Peptide specral matches were filtered using Percolator with a dCN=0.05 and FDR of 1%.

### Statistical Analysis

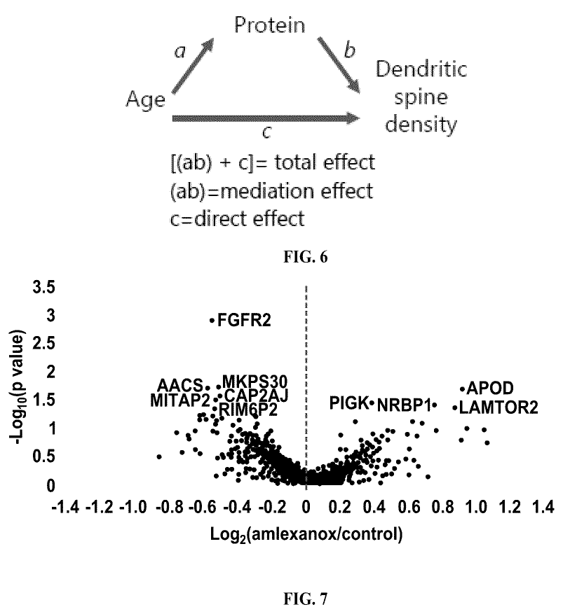

The mean dendritic spine density for each spine size category for each subject was calculated and correlated with each subject's age using linear regression. Peptides were rolled up to protein using inverse-CV-weighted average of scaled peptide values. The effect of age on protein abundance was evaluated with multivariate linear regression with age as the predictor, sex, race, PMI, storage time and plex as covariates, and protein abundance as the outcome. A mediation analysis (FIG. 6) controlling for sex, race, PMI and IHC assay group was conducted to detect proteins which mediated the effect of age on the density of large dendritic spines.

### Results

### Dendritic Spine Density

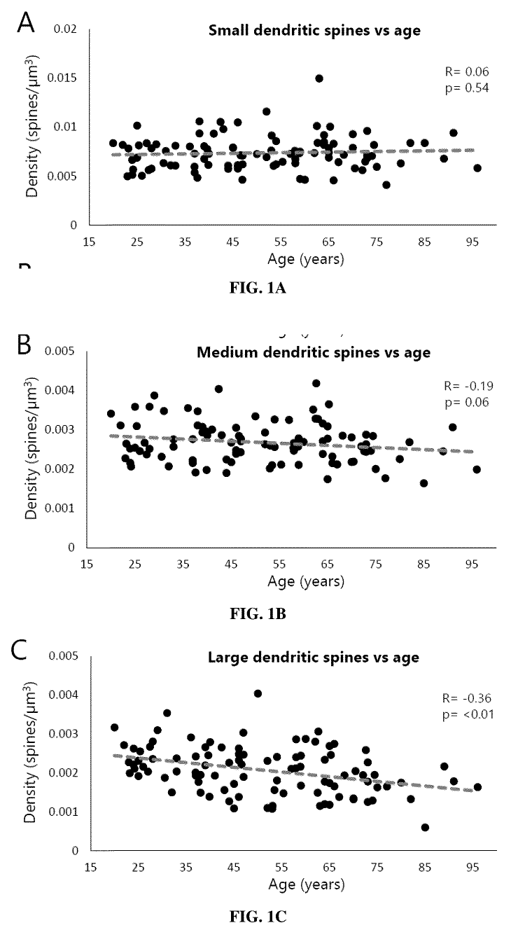

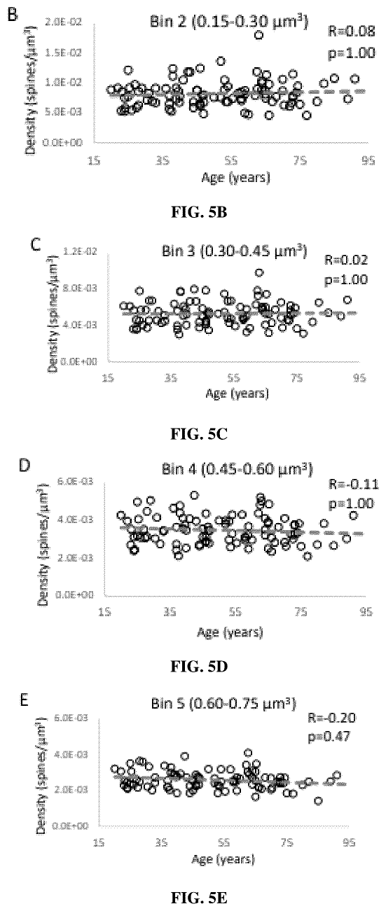

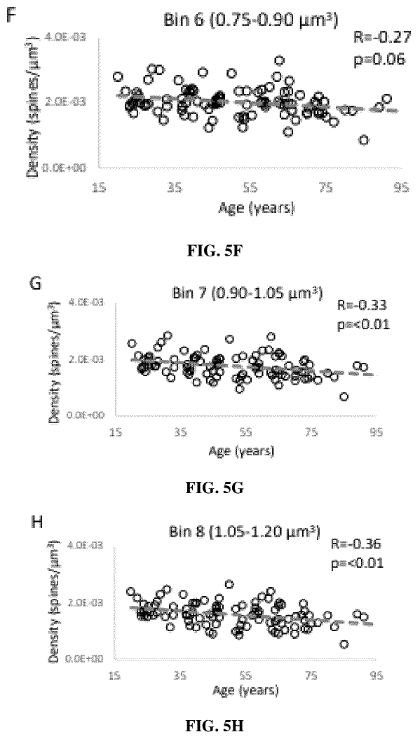

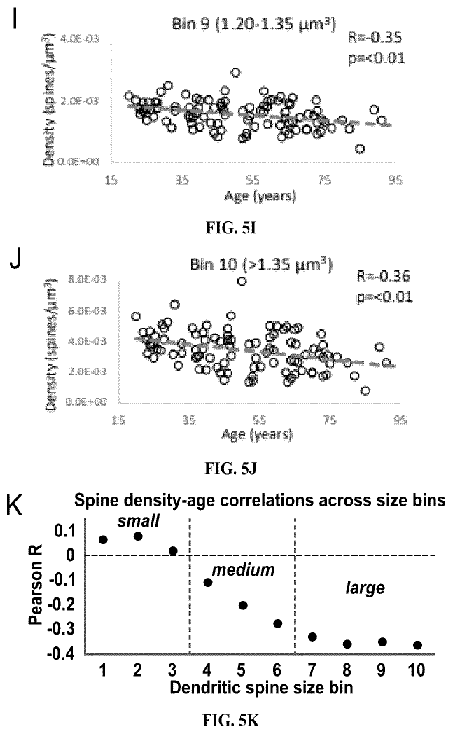

Given that the size morphology of dendritic spines may predict spine stability and contribution to synaptic dynamics[15], dendritic spines were categorized into 10 size bins for correlation with chronological age. Only the density of large dendritic spines (defined by size bins 7-10, volumes >0.90 um³) exhibited robust and significant negative correlations with age (FIG. 1, see also FIG. 5A-5K).

### Homogenate and Synaptosome Proteomics

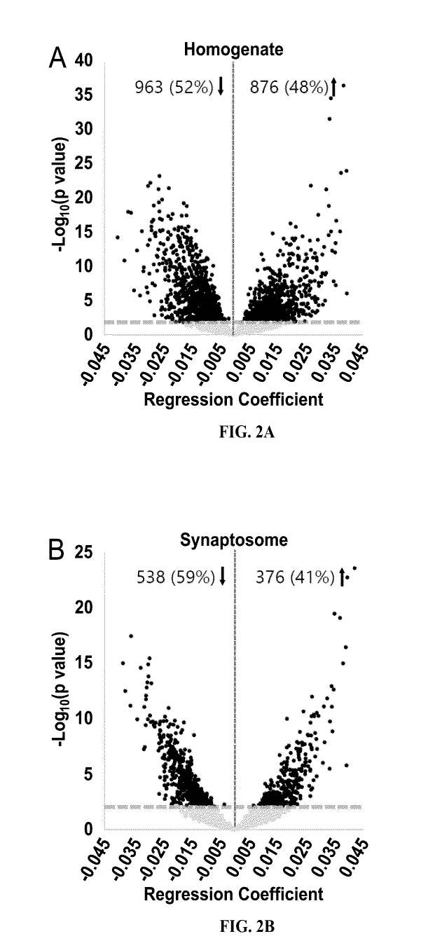

Next the proteome was evaluated within cellular homogenate and synaptosome fractions to identify proteins whose abundance correlated with age. 1839 of 5032 proteins detected in cellular homogenate (˜37%) (FIG. 2A), and 914 of 4754 proteins detected in the synaptosome fraction (˜19%) (FIG. 2B) were significantly correlated with age after adjustment for false discovery. Of the proteins significantly correlated with age, 876 (˜48%) were positively correlated with age in homogenate (Table 5) compared with 376 (41%) proteins positively correlated with age in the synaptosome fraction (Table 6). Relative to 975 proteins which were detected in both cellular compartments, but uniquely correlated with age in the homogenate fraction, only 148 proteins were detected in both cellular compartments but uniquely correlated with age in the synaptosome fraction. Therefore cellular homogenate was the focus as the biological fraction of greatest interest to age-related alterations in the proteome.

Mediation Analyses

A statistical mediation analysis was generated to identify individual homogenate proteins which mediate the effect of age on large dendritic spine density.

For each protein detected in homogenate, the direct effect of age on large dendritic spine density and the mediation effect of age on large dendritic spine density was measured. Lower and upper bootstrapped 95% confidence interval bounds for mediation effects were collected. Proteins which significantly mediate the effect of age on large dendritic spine density were identified (see Table 7).

Table 5 and 6 show the official gene symbols, regression coefficients, regression p values, and regression Q values for all proteins detected in homogenate (Table 5) and synaptosome (Table 6) fractions. Proteins whose abundance values were significantly correlated with age are highlighted in gray. In cellular homogenate, 1839 of 5032 proteins (˜37%) were significantly correlated with age. In the synaptosome fraction, 914 of 4754 proteins (˜19%) were significantly correlated with age. Table 7 show the official gene symbols, the direct effects of age, and their mediation of the effects of age on large dendritic spine density. For each protein detected in homogenate, the direct effect of age on large dendritic spine density and the mediation effect of age on large dendritic spine density is shown. Lower and upper bootstrapped 95% confidence interval bounds for mediation effects are shown. Proteins which significantly mediate the effect of age on large dendritic spine density are highlighted gray.

A total of 203 proteins significantly mediated the effect of age on large dendritic spine density (Table 3A). Each mediator protein was then classified according to the direction of its change with age (increase or decrease) and its relationship to large dendritic spine density (as protein increases spine density increases or as protein increases spine density decreases). The resultant 4 classifications of mediator proteins are shown in Table 3B-3E.

TABLE 3A
Lists all 203 mediator proteins, each of which is a potential drug target.

| | | Mediation | Effect of | Effect of | |
|---|---|---|---|---|---|
| | mediation | Significance | age on | protein | Age |
| Protein | effect | (1 = significant) | protein | on DSD | Coefficient |
| AARS1 | −3.31E−06 | 1 | 0.008205 | −0.0004 | 0.009066 |
| ABRAXAS2 | −2.32E−06 | 1 | −0.00905 | 0.000257 | −0.00735 |
| ACAN | −5.45E−06 | 1 | 0.028329 | −0.00019 | 0.028997 |
| ACSS3 | 4.03E−06 | 1 | 0.020348 | 0.000198 | 0.023192 |
| ADAM22 | 2.29E−06 | 1 | −0.0066 | −0.00035 | −0.00672 |
| ADGRB2 | −5.26E−06 | 1 | −0.02459 | 0.000214 | −0.0239 |
| ALDH1A3 | −5.56E−06 | 1 | 0.027055 | −0.00021 | 0.029336 |
| ALDOC | −3.78E−06 | 1 | 0.009419 | −0.0004 | 0.009087 |
| ANO8 | −3.37E−06 | 1 | 0.018323 | −0.00018 | 0.018956 |
| ANXA11 | −2.10E−06 | 1 | 0.00584 | −0.00036 | 0.005396 |
| AP1S1 | −5.83E−06 | 1 | −0.01893 | 0.000308 | −0.01781 |
| ARHGAP26 | −2.71E−06 | 1 | 0.00592 | −0.00046 | 0.005914 |
| ARL10 | −3.16E−06 | 1 | −0.01403 | 0.000225 | −0.01767 |
| ARMC10 | 2.65E−06 | 1 | 0.008298 | 0.000319 | 0.008267 |
| ARPC5L | −5.13E−06 | 1 | −0.01754 | 0.000293 | −0.01663 |
| ARPP19 | −2.35E−06 | 1 | −0.0117 | 0.000201 | −0.01078 |
| ATG101 | −4.63E−06 | 1 | −0.01731 | 0.000268 | −0.01684 |
| ATG3 | −3.29E−06 | 1 | 0.007827 | −0.00042 | 0.008474 |
| ATP5MD | −3.35E−06 | 1 | −0.01757 | 0.00019 | −0.01514 |
| AZGP1 | −2.98E−06 | 1 | 0.015622 | −0.00019 | 0.014448 |
| BAIAP2 | −7.42E−06 | 1 | −0.01943 | 0.000382 | −0.01894 |
| BLVRB | 1.79E−06 | 1 | 0.009239 | 0.000194 | 0.009847 |
| BSN | −4.91E−06 | 1 | −0.01809 | 0.000272 | −0.01733 |
| BUD13 | −2.26E−06 | 1 | 0.010271 | −0.00022 | 0.008035 |
| C1orf43 | −2.59E−06 | 1 | 0.01269 | −0.0002 | 0.010914 |
| CA11 | −4.38E−06 | 1 | −0.02349 | 0.000186 | −0.02038 |
| CACNG3 | −8.39E−06 | 1 | −0.02126 | 0.000395 | −0.02106 |
| CAMK2G | −3.76E−06 | 1 | 0.01056 | −0.00036 | 0.010932 |
| CAMK4 | −8.50E−06 | 1 | −0.0268 | 0.000317 | −0.02616 |
| CAMKV | −5.01E−06 | 1 | −0.01149 | 0.000436 | −0.01123 |
| CAPG | 2.16E−06 | 1 | 0.014199 | 0.000152 | 0.014002 |
| CAPN5 | −3.16E−06 | 1 | −0.01266 | 0.000249 | −0.01193 |
| CASK | −4.11E−06 | 1 | −0.01652 | 0.000249 | −0.01643 |
| CBARP | −4.83E−06 | 1 | −0.02562 | 0.000189 | −0.02468 |
| CCDC124 | −1.98E−06 | 1 | −0.01138 | 0.000174 | −0.01211 |
| CDK16 | −2.25E−06 | 1 | −0.01338 | 0.000168 | −0.01062 |
| CELF5 | −2.51E−06 | 1 | −0.01633 | 0.000154 | −0.01541 |
| CHD4 | −2.22E−06 | 1 | 0.010103 | −0.00022 | 0.009134 |
| CHM | −2.58E−06 | 1 | −0.00987 | 0.000261 | −0.00868 |
| CLPB | 1.93E−06 | 1 | 0.007714 | 0.00025 | 0.008847 |
| CNN3 | 3.57E−06 | 1 | 0.01499 | 0.000238 | 0.015764 |
| COMMD3 | −2.63E−06 | 1 | 0.012109 | −0.00022 | 0.009661 |
| CPLX3 | −5.03E−06 | 1 | −0.03261 | 0.000154 | −0.03143 |
| CRIP2 | −5.24E−06 | 1 | −0.02387 | 0.00022 | −0.02229 |
| CRMP1 | −5.13E−06 | 1 | −0.01502 | 0.000341 | −0.0146 |
| CSDC2 | −4.75E−06 | 1 | −0.01516 | 0.000314 | −0.01054 |
| CSDE1 | 2.05E−06 | 1 | −0.0054 | −0.00038 | −0.00534 |
| CSNK2A2 | 2.32E−06 | 1 | −0.00601 | −0.00039 | −0.00789 |
| CXADR | −6.33E−06 | 1 | −0.02868 | 0.000221 | −0.02721 |
| CYB5R3 | 2.88E−06 | 1 | 0.007969 | 0.000361 | 0.008149 |
| CYP46A1 | −6.15E−06 | 1 | −0.03063 | 0.000201 | −0.02986 |
| DGKB | −4.92E−06 | 1 | −0.0121 | 0.000407 | −0.01159 |
| DHRS11 | −2.42E−06 | 1 | 0.013022 | −0.00019 | 0.014645 |
| DIS3L2 | 1.87E−06 | 1 | −0.01066 | −0.00018 | −0.01111 |
| DLGAP1 | −8.06E−06 | 1 | −0.01056 | 0.000764 | −0.00982 |
| DMTN | −2.41E−06 | 1 | −0.01075 | 0.000224 | −0.01056 |
| DNAJC19 | 1.26E−06 | 1 | 0.009082 | 0.000139 | 0.007508 |
| DOC2A | −2.71E−06 | 1 | −0.01087 | 0.000249 | −0.00946 |
| EEF1E1 | −1.97E−06 | 1 | −0.0105 | 0.000187 | −0.00844 |
| EIF2B1 | 2.06E−06 | 1 | −0.00642 | −0.00032 | −0.00545 |
| EIF2S1 | 1.95E−06 | 1 | −0.0055 | −0.00035 | −0.00598 |
| EIF3I | −2.07E−06 | 1 | −0.00813 | 0.000255 | −0.00689 |
| EML2 | −2.58E−06 | 1 | 0.015948 | −0.00016 | 0.016437 |
| ENDOD1 | −4.13E−06 | 1 | 0.018352 | −0.00022 | 0.018042 |
| ENPP6 | 2.52E−06 | 1 | −0.01482 | −0.00017 | −0.01518 |
| ENSA | −3.39E−06 | 1 | −0.00967 | 0.000351 | −0.00744 |
| EPB41L3 | −3.82E−06 | 1 | 0.009439 | −0.00041 | 0.009287 |
| EPHB2 | 5.69E−06 | 1 | −0.01864 | −0.00031 | −0.01871 |
| ESYT2 | −2.99E−06 | 1 | 0.008881 | −0.00034 | 0.008855 |
| ETF1 | 2.63E−06 | 1 | −0.00623 | −0.00042 | −0.00705 |
| EXOC4 | −2.09E−06 | 1 | −0.00449 | 0.000465 | −0.00411 |
| FGF1 | −2.89E−06 | 1 | 0.016229 | −0.00018 | 0.01446 |
| FKBP4 | −3.19E−06 | 1 | 0.007182 | −0.00044 | 0.006829 |
| FKBP5 | −1.06E−05 | 1 | 0.034846 | −0.0003 | 0.034096 |
| FNBP1 | −4.92E−06 | 1 | 0.015034 | −0.00033 | 0.015275 |
| FOLH1 | −8.04E−06 | 1 | 0.02918 | −0.00028 | 0.027611 |
| FRRS1L | −3.21E−06 | 1 | −0.00968 | 0.000331 | −0.00859 |
| FSD1 | 6.30E−06 | 1 | −0.01063 | −0.00059 | −0.01059 |
| GPD1 | 1.65E−06 | 1 | −0.01061 | −0.00016 | −0.01134 |
| GRIN1 | −6.48E−06 | 1 | −0.01368 | 0.000473 | −0.0131 |
| H1-5 | −3.20E−06 | 1 | 0.020497 | −0.00016 | 0.018927 |
| HMGCS1 | −5.38E−06 | 1 | −0.01798 | 0.000299 | −0.01775 |
| HNRNPM | −1.81E−06 | 1 | 0.005287 | −0.00034 | 0.004947 |
| HSPA4L | −3.97E−06 | 1 | 0.008714 | −0.00046 | 0.009379 |
| HSPE1 | −2.97E−06 | 1 | 0.011109 | −0.00027 | 0.01136 |
| HSPH1 | 3.97E−06 | 1 | −0.00827 | −0.00048 | −0.00874 |
| IQGAP1 | −3.48E−06 | 1 | 0.020308 | −0.00017 | 0.020417 |
| KALRN | −1.06E−05 | 1 | −0.0213 | 0.000497 | −0.02181 |
| KIF16B | −5.36E−06 | 1 | −0.02331 | 0.00023 | −0.02241 |
| KIF3A | −6.98E−06 | 1 | −0.01378 | 0.000506 | −0.01297 |
| LASP1 | −7.63E−06 | 1 | −0.02007 | 0.00038 | −0.01926 |
| LGALS3BP | −2.21E−06 | 1 | 0.013839 | −0.00016 | 0.015012 |
| LGI1 | 2.52E−06 | 1 | −0.0089 | −0.00028 | −0.00863 |
| LIMK1 | −2.26E−06 | 1 | 0.010045 | −0.00022 | 0.010559 |
| LLGL1 | −4.41E−06 | 1 | 0.021752 | −0.0002 | 0.022349 |
| LMBRD2 | −2.03E−06 | 1 | −0.00683 | 0.000298 | −0.0079 |
| LRRC73 | −3.27E−06 | 1 | −0.01759 | 0.000186 | −0.01543 |
| MAL2 | −3.22E−06 | 1 | −0.02332 | 0.000138 | −0.02293 |
| MAP2K7 | −1.69E−06 | 1 | 0.014274 | −0.00012 | 0.013107 |
| MAPK10 | −3.77E−06 | 1 | −0.00887 | 0.000425 | −0.00745 |
| MAPT | −6.31E−06 | 1 | −0.01284 | 0.000491 | −0.0123 |
| MGST1 | 2.24E−06 | 1 | 0.011545 | 0.000194 | 0.011492 |
| MID1IP1 | −5.44E−06 | 1 | 0.022628 | −0.00024 | 0.022715 |
| MIOS | −2.39E−06 | 1 | 0.011998 | −0.0002 | 0.011786 |
| MOB2 | −2.99E−06 | 1 | 0.009292 | −0.00032 | 0.008846 |
| MPP1 | −4.95E−06 | 1 | 0.021706 | −0.00023 | 0.022548 |
| MRVI1 | −4.47E−06 | 1 | 0.024995 | −0.00018 | 0.024493 |
| MTPN | −3.06E−06 | 1 | −0.01383 | 0.000221 | −0.01413 |
| NANS | −2.77E−06 | 1 | 0.007694 | −0.00036 | 0.00808 |
| NCKIPSD | −8.72E−06 | 1 | 0.019679 | −0.00044 | 0.019932 |
| NEBL | −3.73E−06 | 1 | 0.01437 | −0.00026 | 0.013124 |
| NMT2 | −4.27E−06 | 1 | 0.014915 | −0.00029 | 0.013868 |
| NT5C2 | −1.95E−06 | 1 | 0.00862 | −0.00023 | 0.006815 |
| NTNG2 | −5.78E−06 | 1 | −0.02366 | 0.000244 | −0.01691 |
| NUDT5 | −2.89E−06 | 1 | 0.014231 | −0.0002 | 0.015004 |
| OSBPL1A | 1.94E−06 | 1 | −0.00591 | −0.00033 | −0.00622 |
| PAK3 | −4.14E−06 | 1 | −0.00816 | 0.000506 | −0.00727 |
| PATJ | −4.97E−06 | 1 | 0.018454 | −0.00027 | 0.027123 |
| PBXIP1 | −4.88E−06 | 1 | 0.025983 | −0.00019 | 0.024957 |
| PCBD1 | 2.79E−06 | 1 | 0.018261 | 0.000153 | 0.020379 |
| PCDH10 | 2.87E−06 | 1 | −0.01838 | −0.00016 | −0.01838 |
| PDLIM5 | −4.60E−06 | 1 | 0.021229 | −0.00022 | 0.019525 |
| PDZD4 | −6.12E−06 | 1 | −0.01681 | 0.000364 | −0.01237 |
| PFKM | −2.44E−06 | 1 | 0.006602 | −0.00037 | 0.007385 |
| PIANP | −6.69E−06 | 1 | −0.02477 | 0.00027 | −0.02289 |
| PIN1 | 1.88E−06 | 1 | −0.00759 | −0.00025 | −0.00737 |
| PIPOX | −3.11E−06 | 1 | 0.015641 | −0.0002 | 0.01191 |
| PITHD1 | −3.16E−06 | 1 | 0.006545 | −0.00048 | 0.00484 |
| PKM | −5.49E−06 | 1 | 0.008354 | −0.00066 | 0.008399 |
| PLCH2 | −4.08E−06 | 1 | −0.02148 | 0.00019 | −0.01634 |
| PLD3 | −2.49E−06 | 1 | −0.00695 | 0.000359 | −0.00723 |
| PPM1H | −1.64E−06 | 1 | −0.00512 | 0.00032 | −0.00499 |
| PPP1R1B | 2.72E−06 | 1 | 0.013826 | 0.000196 | 0.015378 |
| PPP2R2D | 2.18E−06 | 1 | −0.01081 | −0.0002 | −0.00976 |
| PPP2R5D | 1.82E−06 | 1 | −0.00355 | −0.00051 | −0.00315 |
| PRKAR1A | −2.52E−06 | 1 | 0.01035 | −0.00024 | 0.010807 |
| PRKAR2A | 2.19E−06 | 1 | −0.00651 | −0.00034 | −0.00604 |
| PRPF31 | −1.94E−06 | 1 | 0.01034 | −0.00019 | 0.01096 |
| PSD2 | −3.61E−06 | 1 | 0.019838 | −0.00018 | 0.01898 |
| PSMC2 | −2.09E−06 | 1 | −0.00365 | 0.000573 | −0.00304 |
| PSMD1 | −1.87E−06 | 1 | −0.00459 | 0.000406 | −0.00407 |
| PSME2 | 3.02E−06 | 1 | 0.010531 | 0.000287 | 0.011649 |
| PTK2B | −4.38E−06 | 1 | 0.012017 | −0.00036 | 0.0125 |
| PTPRT | −2.09E−06 | 1 | 0.008626 | −0.00024 | 0.007752 |
| PURG | −7.92E−06 | 1 | −0.02822 | 0.000281 | −0.02718 |
| QARS1 | −2.20E−06 | 1 | −0.00711 | 0.000309 | −0.00727 |
| RAB23 | 2.71E−06 | 1 | 0.00524 | 0.000518 | 0.005989 |
| RAB9A | 2.60E−06 | 1 | 0.017748 | 0.000147 | 0.020204 |
| RAB9B | 2.83E−06 | 1 | 0.01563 | 0.000181 | 0.015572 |
| RAD23B | −3.78E−06 | 1 | −0.01017 | 0.000372 | −0.01084 |
| RASAL1 | −6.09E−06 | 1 | −0.02306 | 0.000264 | −0.02273 |
| REEP1 | −1.91E−06 | 1 | −0.00832 | 0.000229 | −0.00915 |
| RELCH | 3.66E−06 | 1 | −0.01111 | −0.00033 | −0.0118 |
| REPS1 | −5.06E−06 | 1 | −0.01187 | 0.000426 | −0.00991 |
| RGS17 | −3.64E−06 | 1 | −0.01546 | 0.000236 | −0.01267 |
| RPL13 | −2.08E−06 | 1 | −0.00744 | 0.000279 | −0.0072 |
| RPL7 | −1.66E−06 | 1 | −0.00681 | 0.000243 | −0.00702 |
| RPS6KA5 | −7.10E−06 | 1 | 0.032574 | −0.00022 | 0.03169 |
| RPS9 | −1.98E−06 | 1 | −0.00732 | 0.00027 | −0.00811 |
| RSU1 | 1.53E−06 | 1 | 0.010479 | 0.000146 | 0.013574 |
| RTN1 | −2.24E−06 | 1 | −0.008 | 0.00028 | −0.00806 |
| S100A6 | 2.81E−06 | 1 | 0.023201 | 0.000121 | 0.021908 |
| S100A8 | −3.32E−06 | 1 | 0.017637 | −0.00019 | 0.015691 |
| S100A9 | −2.56E−06 | 1 | 0.015999 | −0.00016 | 0.014342 |
| SASH1 | −2.03E−06 | 1 | 0.013762 | −0.00015 | 0.013656 |
| SCG2 | 3.41E−06 | 1 | 0.016391 | 0.000208 | 0.016111 |
| SCN2A | −1.53E−06 | 1 | −0.00601 | 0.000254 | −0.00657 |
| SEPTIN10 | −3.20E−06 | 1 | 0.023198 | −0.00014 | 0.023795 |
| SERINC1 | 1.77E−06 | 1 | 0.006336 | 0.000279 | 0.008901 |
| SLC17A7 | −3.97E−06 | 1 | −0.01097 | 0.000362 | −0.01075 |
| SLC25A1 | 3.18E−06 | 1 | 0.013983 | 0.000227 | 0.01538 |
| SLC30A1 | −2.17E−06 | 1 | −0.00648 | 0.000334 | −0.00591 |
| SLC4A10 | −5.50E−06 | 1 | −0.01238 | 0.000444 | −0.01124 |
| SLC7A5 | 5.62E−06 | 1 | 0.012731 | 0.000441 | 0.01484 |
| SMIM13 | −3.11E−06 | 1 | −0.01538 | 0.000202 | −0.01658 |
| SNCA | −6.41E−06 | 1 | −0.01475 | 0.000435 | −0.01479 |
| SNX12 | −3.73E−06 | 1 | 0.008765 | −0.00043 | 0.006978 |
| SPART | −6.78E−06 | 1 | 0.016991 | −0.0004 | 0.016283 |
| SRSF7 | −1.90E−06 | 1 | 0.00603 | −0.00032 | 0.003097 |
| STK32C | −5.04E−06 | 1 | −0.01459 | 0.000345 | −0.0139 |
| STMN1 | −3.37E−06 | 1 | −0.01336 | 0.000253 | −0.01263 |
| STX12 | −2.76E−06 | 1 | −0.00799 | 0.000346 | −0.00826 |
| SWAP70 | −2.65E−06 | 1 | 0.012245 | −0.00022 | 0.011642 |
| SYN1 | −2.09E−06 | 1 | −0.00753 | 0.000278 | −0.00651 |
| SYN2 | −2.54E−06 | 1 | −0.00844 | 0.000301 | −0.0083 |
| SYNGR1 | −1.77E−06 | 1 | −0.01392 | 0.000127 | −0.01481 |
| SYT5 | −3.70E−06 | 1 | −0.01587 | 0.000233 | −0.01444 |
| TMC6 | −2.46E−06 | 1 | 0.017276 | −0.00014 | 0.017928 |
| TMEM163 | −3.55E−06 | 1 | −0.0122 | 0.000291 | −0.00971 |
| TMEM63B | −2.26E−06 | 1 | −0.00938 | 0.000241 | −0.00866 |
| TPM1 | 1.86E−06 | 1 | 0.006982 | 0.000266 | 0.007936 |
| TPP2 | 2.57E−06 | 1 | −0.00875 | −0.00029 | −0.00847 |
| TRIO | −7.01E−06 | 1 | −0.01537 | 0.000456 | −0.01488 |
| TSPAN15 | −3.13E−06 | 1 | 0.01804 | −0.00017 | 0.011778 |
| TSPAN7 | −1.51E−06 | 1 | −0.00556 | 0.000271 | −0.00554 |
| TTPAL | −4.87E−06 | 1 | −0.02183 | 0.000223 | −0.02213 |
| UBAP1 | −3.00E−06 | 1 | 0.011945 | −0.00025 | 0.010648 |
| UBE2I | 2.05E−06 | 1 | 0.012918 | 0.000159 | 0.012592 |
| UBE3A | −4.15E−06 | 1 | −0.00922 | 0.00045 | −0.00827 |
| VPS53 | 1.88E−06 | 1 | −0.00611 | −0.00031 | −0.0061 |
| WDR54 | 1.77E−06 | 1 | −0.01153 | −0.00015 | −0.01057 |
| XKR4 | −1.10E−05 | 1 | −0.0408 | 0.00027 | −0.04043 |
| ZNF706 | −3.05E−06 | 1 | −0.0143 | 0.000213 | −0.01158 |

TABLE 3B
List of mediator proteins based on the protein levels increase with increasing
age and increased protein levels are associated with higher spine densities.

| | | Mediation | Effect of | Effect of |
|---|---|---|---|---|
| | mediation | Significance | age on | protein |
| Protein | effect | (1 = significant) | protein | on DSD |
| S100A6 | 2.81E−06 | 1 | 0.023201 | 0.000121 |
| ACSS3 | 4.03E−06 | 1 | 0.020348 | 0.000198 |
| PCBD1 | 2.79E−06 | 1 | 0.018261 | 0.000153 |
| RAB9A | 2.60E−06 | 1 | 0.017748 | 0.000147 |
| SCG2 | 3.41E−06 | 1 | 0.016391 | 0.000208 |
| RAB9B | 2.83E−06 | 1 | 0.01563 | 0.000181 |
| CNN3 | 3.57E−06 | 1 | 0.01499 | 0.000238 |
| CAPG | 2.16E−06 | 1 | 0.014199 | 0.000152 |
| SLC25A1 | 3.18E−06 | 1 | 0.013983 | 0.000227 |
| PPP1R1B | 2.72E−06 | 1 | 0.013826 | 0.000196 |
| UBE2I | 2.05E−06 | 1 | 0.012918 | 0.000159 |
| SLC7A5 | 5.62E−06 | 1 | 0.012731 | 0.000441 |
| MGST1 | 2.24E−06 | 1 | 0.011545 | 0.000194 |
| PSME2 | 3.02E−06 | 1 | 0.010531 | 0.000287 |
| RSU1 | 1.53E−06 | 1 | 0.010479 | 0.000146 |
| BLVRB | 1.79E−06 | 1 | 0.009239 | 0.000194 |
| DNAJC19 | 1.26E−06 | 1 | 0.009082 | 0.000139 |
| ARMC10 | 2.65E−06 | 1 | 0.008298 | 0.000319 |
| CYB5R3 | 2.88E−06 | 1 | 0.007969 | 0.000361 |
| CLPB | 1.93E−06 | 1 | 0.007714 | 0.00025 |
| TPM1 | 1.86E−06 | 1 | 0.006982 | 0.000266 |
| SERINC1 | 1.77E−06 | 1 | 0.006336 | 0.000279 |
| RAB23 | 2.71E−06 | 1 | 0.00524 | 0.000518 |

TABLE 3C
List of mediator proteins based on the protein levels increase with increasing
age and decreased protein levels are associated with higher spine densities.

| | | Mediation | Effect of | Effect of |
|---|---|---|---|---|
| | mediation | Significance | age on | protein on |
| Protein | effect | (1 = significant) | protein | DSD |
| FKBP5 | −1.06E−05 | 1 | 0.034846 | −0.0003 |
| RPS6KA5 | −7.10E−06 | 1 | 0.032574 | −0.00022 |
| FOLH1 | −8.04E−06 | 1 | 0.02918 | −0.00028 |
| ACAN | −5.45E−06 | 1 | 0.028329 | −0.00019 |
| ALDH1A3 | −5.56E−06 | 1 | 0.027055 | −0.00021 |
| PBXIP1 | −4.88E−06 | 1 | 0.025983 | −0.00019 |
| MRVI1 | −4.47E−06 | 1 | 0.024995 | −0.00018 |
| SEPTIN10 | −3.20E−06 | 1 | 0.023198 | −0.00014 |
| MID1IP1 | −5.44E−06 | 1 | 0.022628 | −0.00024 |
| LLGL1 | −4.41E−06 | 1 | 0.021752 | −0.0002 |
| MPP1 | −4.95E−06 | 1 | 0.021706 | −0.00023 |
| PDLIM5 | −4.60E−06 | 1 | 0.021229 | −0.00022 |
| H1-5 | −3.20E−06 | 1 | 0.020497 | −0.00016 |
| IQGAP1 | −3.48E−06 | 1 | 0.020308 | −0.00017 |
| PSD2 | −3.61E−06 | 1 | 0.019838 | −0.00018 |
| NCKIPSD | −8.72E−06 | 1 | 0.019679 | −0.00044 |
| PATJ | −4.97E−06 | 1 | 0.018454 | −0.00027 |
| ENDOD1 | −4.13E−06 | 1 | 0.018352 | −0.00022 |
| ANO8 | −3.37E−06 | 1 | 0.018323 | −0.00018 |
| TSPAN15 | −3.13E−06 | 1 | 0.01804 | −0.00017 |
| S100A8 | −3.32E−06 | 1 | 0.017637 | −0.00019 |
| TMC6 | −2.46E−06 | 1 | 0.017276 | −0.00014 |
| SPART | −6.78E−06 | 1 | 0.016991 | −0.0004 |
| FGF1 | −2.89E−06 | 1 | 0.016229 | −0.00018 |
| S100A9 | −2.56E−06 | 1 | 0.015999 | −0.00016 |
| EML2 | −2.58E−06 | 1 | 0.015948 | −0.00016 |
| PIPOX | −3.11E−06 | 1 | 0.015641 | −0.0002 |
| AZGP1 | −2.98E−06 | 1 | 0.015622 | −0.00019 |
| FNBP1 | −4.92E−06 | 1 | 0.015034 | −0.00033 |
| NMT2 | −4.27E−06 | 1 | 0.014915 | −0.00029 |
| NEBL | −3.73E−06 | 1 | 0.01437 | −0.00026 |
| MAP2K7 | −1.69E−06 | 1 | 0.014274 | −0.00012 |
| NUDT5 | −2.89E−06 | 1 | 0.014231 | −0.0002 |
| LGALS3BP | −2.21E−06 | 1 | 0.013839 | −0.00016 |
| SASH1 | −2.03E−06 | 1 | 0.013762 | −0.00015 |
| DHRS11 | −2.42E−06 | 1 | 0.013022 | −0.00019 |
| C1orf43 | −2.59E−06 | 1 | 0.01269 | −0.0002 |
| SWAP70 | −2.65E−06 | 1 | 0.012245 | −0.00022 |
| COMMD3 | −2.63E−06 | 1 | 0.012109 | −0.00022 |
| PTK2B | −4.38E−06 | 1 | 0.012017 | −0.00036 |
| MIOS | −2.39E−06 | 1 | 0.011998 | −0.0002 |
| UBAP1 | −3.00E−06 | 1 | 0.011945 | −0.00025 |
| HSPE1 | −2.97E−06 | 1 | 0.011109 | −0.00027 |
| CAMK2G | −3.76E−06 | 1 | 0.01056 | −0.00036 |
| PRKAR1A | −2.52E−06 | 1 | 0.01035 | −0.00024 |
| PRPF31 | −1.94E−06 | 1 | 0.01034 | −0.00019 |
| BUD13 | −2.26E−06 | 1 | 0.010271 | −0.00022 |
| CHD4 | −2.22E−06 | 1 | 0.010103 | −0.00022 |
| LIMK1 | −2.26E−06 | 1 | 0.010045 | −0.00022 |
| EPB41L3 | −3.82E−06 | 1 | 0.009439 | −0.00041 |
| ALDOC | −3.78E−06 | 1 | 0.009419 | −0.0004 |
| MOB2 | −2.99E−06 | 1 | 0.009292 | −0.00032 |
| ESYT2 | −2.99E−06 | 1 | 0.008881 | −0.00034 |
| SNX12 | −3.73E−06 | 1 | 0.008765 | −0.00043 |
| HSPA4L | −3.97E−06 | 1 | 0.008714 | −0.00046 |
| PTPRT | −2.09E−06 | 1 | 0.008626 | −0.00024 |
| NT5C2 | −1.95E−06 | 1 | 0.00862 | −0.00023 |
| PKM | −5.49E−06 | 1 | 0.008354 | −0.00066 |
| AARS1 | −3.31E−06 | 1 | 0.008205 | −0.0004 |
| ATG3 | −3.29E−06 | 1 | 0.007827 | −0.00042 |
| NANS | −2.77E−06 | 1 | 0.007694 | −0.00036 |
| FKBP4 | −3.19E−06 | 1 | 0.007182 | −0.00044 |
| PFKM | −2.44E−06 | 1 | 0.006602 | −0.00037 |
| PITHD1 | −3.16E−06 | 1 | 0.006545 | −0.00048 |
| SRSF7 | −1.90E−06 | 1 | 0.00603 | −0.00032 |
| ARHGAP26 | −2.71E−06 | 1 | 0.00592 | −0.00046 |
| ANXA11 | −2.10E−06 | 1 | 0.00584 | −0.00036 |
| HNRNPM | −1.81E−06 | 1 | 0.005287 | −0.00034 |

TABLE 3D
List of mediator proteins based on the protein levels decrease with increasing
age and increased protein levels are associated with higher spine densities.

| | | Mediation | Effect of | Effect of |
|---|---|---|---|---|
| | mediation | Significance | age on | protein on |
| Protein | effect | (1 = significant) | protein | DSD |
| XKR4 | −1.10E−05 | 1 | −0.0408 | 0.00027 |
| CPLX3 | −5.03E−06 | 1 | −0.03261 | 0.000154 |
| CYP46A1 | −6.15E−06 | 1 | −0.03063 | 0.000201 |
| CXADR | −6.33E−06 | 1 | −0.02868 | 0.000221 |
| PURG | −7.92E−06 | 1 | −0.02822 | 0.000281 |
| CAMK4 | −8.50E−06 | 1 | −0.0268 | 0.000317 |
| CBARP | −4.83E−06 | 1 | −0.02562 | 0.000189 |
| PIANP | −6.69E−06 | 1 | −0.02477 | 0.00027 |
| ADGRB2 | −5.26E−06 | 1 | −0.02459 | 0.000214 |
| CRIP2 | −5.24E−06 | 1 | −0.02387 | 0.00022 |
| NTNG2 | −5.78E−06 | 1 | −0.02366 | 0.000244 |
| CA11 | −4.38E−06 | 1 | −0.02349 | 0.000186 |
| MAL2 | −3.22E−06 | 1 | −0.02332 | 0.000138 |
| KIF16B | −5.36E−06 | 1 | −0.02331 | 0.00023 |
| RASAL1 | −6.09E−06 | 1 | −0.02306 | 0.000264 |
| TTPAL | −4.87E−06 | 1 | −0.02183 | 0.000223 |
| PLCH2 | −4.08E−06 | 1 | −0.02148 | 0.00019 |
| KALRN | −1.06E−05 | 1 | −0.0213 | 0.000497 |
| CACNG3 | −8.39E−06 | 1 | −0.02126 | 0.000395 |
| LASP1 | −7.63E−06 | 1 | −0.02007 | 0.00038 |
| BAIAP2 | −7.42E−06 | 1 | −0.01943 | 0.000382 |
| AP1S1 | −5.83E−06 | 1 | −0.01893 | 0.000308 |
| BSN | −4.91E−06 | 1 | −0.01809 | 0.000272 |
| HMGCS1 | −5.38E−06 | 1 | −0.01798 | 0.000299 |
| LRRC73 | −3.27E−06 | 1 | −0.01759 | 0.000186 |
| ATP5MD | −3.35E−06 | 1 | −0.01757 | 0.00019 |
| ARPC5L | −5.13E−06 | 1 | −0.01754 | 0.000293 |
| ATG101 | −4.63E−06 | 1 | −0.01731 | 0.000268 |
| PDZD4 | −6.12E−06 | 1 | −0.01681 | 0.000364 |
| CASK | −4.11E−06 | 1 | −0.01652 | 0.000249 |
| CELF5 | −2.51E−06 | 1 | −0.01633 | 0.000154 |
| SYT5 | −3.70E−06 | 1 | −0.01587 | 0.000233 |
| RGS17 | −3.64E−06 | 1 | −0.01546 | 0.000236 |
| SMIM13 | −3.11E−06 | 1 | −0.01538 | 0.000202 |
| TRIO | −7.01E−06 | 1 | −0.01537 | 0.000456 |
| CSDC2 | −4.75E−06 | 1 | −0.01516 | 0.000314 |
| CRMP1 | −5.13E−06 | 1 | −0.01502 | 0.000341 |
| SNCA | −6.41E−06 | 1 | −0.01475 | 0.000435 |
| STK32C | −5.04E−06 | 1 | −0.01459 | 0.000345 |
| ZNF706 | −3.05E−06 | 1 | −0.0143 | 0.000213 |
| ARL10 | −3.16E−06 | 1 | −0.01403 | 0.000225 |
| SYNGR1 | −1.77E−06 | 1 | −0.01392 | 0.000127 |
| MTPN | −3.06E−06 | 1 | −0.01383 | 0.000221 |
| KIF3A | −6.98E−06 | 1 | −0.01378 | 0.000506 |
| GRIN1 | −6.48E−06 | 1 | −0.01368 | 0.000473 |
| CDK16 | −2.25E−06 | 1 | −0.01338 | 0.000168 |
| STMN1 | −3.37E−06 | 1 | −0.01336 | 0.000253 |
| MAPT | −6.31E−06 | 1 | −0.01284 | 0.000491 |
| CAPN5 | −3.16E−06 | 1 | −0.01266 | 0.000249 |
| SLC4A10 | −5.50E−06 | 1 | −0.01238 | 0.000444 |
| TMEM163 | −3.55E−06 | 1 | −0.0122 | 0.000291 |
| DGKB | −4.92E−06 | 1 | −0.0121 | 0.000407 |
| REPS1 | −5.06E−06 | 1 | −0.01187 | 0.000426 |
| ARPP19 | −2.35E−06 | 1 | −0.0117 | 0.000201 |
| CAMKV | −5.01E−06 | 1 | −0.01149 | 0.000436 |
| CCDC124 | −1.98E−06 | 1 | −0.01138 | 0.000174 |
| SLC17A7 | −3.97E−06 | 1 | −0.01097 | 0.000362 |
| DOC2A | −2.71E−06 | 1 | −0.01087 | 0.000249 |
| DMTN | −2.41E−06 | 1 | −0.01075 | 0.000224 |
| DLGAP1 | −8.06E−06 | 1 | −0.01056 | 0.000764 |
| EEF1E1 | −1.97E−06 | 1 | −0.0105 | 0.000187 |
| RAD23B | −3.78E−06 | 1 | −0.01017 | 0.000372 |
| CHM | −2.58E−06 | 1 | −0.00987 | 0.000261 |
| FRRS1L | −3.21E−06 | 1 | −0.00968 | 0.000331 |
| ENSA | −3.39E−06 | 1 | −0.00967 | 0.000351 |
| TMEM63B | −2.26E−06 | 1 | −0.00938 | 0.000241 |
| UBE3A | −4.15E−06 | 1 | −0.00922 | 0.00045 |
| ABRAXAS2 | −2.32E−06 | 1 | −0.00905 | 0.000257 |
| MAPK10 | −3.77E−06 | 1 | −0.00887 | 0.000425 |
| SYN2 | −2.54E−06 | 1 | −0.00844 | 0.000301 |
| REEP1 | −1.91E−06 | 1 | −0.00832 | 0.000229 |
| PAK3 | −4.14E−06 | 1 | −0.00816 | 0.000506 |
| EIF3I | −2.07E−06 | 1 | −0.00813 | 0.000255 |
| RTN1 | −2.24E−06 | 1 | −0.008 | 0.00028 |
| STX12 | −2.76E−06 | 1 | −0.00799 | 0.000346 |
| SYN1 | −2.09E−06 | 1 | −0.00753 | 0.000278 |
| RPL13 | −2.08E−06 | 1 | −0.00744 | 0.000279 |
| RPS9 | −1.98E−06 | 1 | −0.00732 | 0.00027 |
| QARS1 | −2.20E−06 | 1 | −0.00711 | 0.000309 |
| PLD3 | −2.49E−06 | 1 | −0.00695 | 0.000359 |
| LMBRD2 | −2.03E−06 | 1 | −0.00683 | 0.000298 |
| RPL7 | −1.66E−06 | 1 | −0.00681 | 0.000243 |
| SLC30A1 | −2.17E−06 | 1 | −0.00648 | 0.000334 |
| SCN2A | −1.53E−06 | 1 | −0.00601 | 0.000254 |
| TSPAN7 | −1.51E−06 | 1 | −0.00556 | 0.000271 |
| PPM1H | −1.64E−06 | 1 | −0.00512 | 0.00032 |
| PSMD1 | −1.87E−06 | 1 | −0.00459 | 0.000406 |
| EXOC4 | −2.09E−06 | 1 | −0.00449 | 0.000465 |
| PSMC2 | −2.09E−06 | 1 | −0.00365 | 0.000573 |

TABLE 3E
List of mediator proteins based on the protein levels decrease with increasing
age and decreased protein levels are associated with higher spine densities.

| | | Mediation | Effect of | Effect of |
|---|---|---|---|---|
| | mediation | Significance | age on | protein on |
| Protein | effect | (1 = significant) | protein | DSD |
| EPHB2 | 5.69E−06 | 1 | −0.01864 | −0.00031 |
| PCDH10 | 2.87E−06 | 1 | −0.01838 | −0.00016 |
| ENPP6 | 2.52E−06 | 1 | −0.01482 | −0.00017 |
| WDR54 | 1.77E−06 | 1 | −0.01153 | −0.00015 |
| RELCH | 3.66E−06 | 1 | −0.01111 | −0.00033 |
| PPP2R2D | 2.18E−06 | 1 | −0.01081 | −0.0002 |
| DIS3L2 | 1.87E−06 | 1 | −0.01066 | −0.00018 |
| FSD1 | 6.30E−06 | 1 | −0.01063 | −0.00059 |
| GPD1 | 1.65E−06 | 1 | −0.01061 | −0.00016 |
| LGI1 | 2.52E−06 | 1 | −0.0089 | −0.00028 |
| TPP2 | 2.57E−06 | 1 | −0.00875 | −0.00029 |
| HSPH1 | 3.97E−06 | 1 | −0.00827 | −0.00048 |
| PIN1 | 1.88E−06 | 1 | −0.00759 | −0.00025 |
| ADAM22 | 2.29E−06 | 1 | −0.0066 | −0.00035 |
| PRKAR2A | 2.19E−06 | 1 | −0.00651 | −0.00034 |
| EIF2B1 | 2.06E−06 | 1 | −0.00642 | −0.00032 |
| ETF1 | 2.63E−06 | 1 | −0.00623 | −0.00042 |
| VPS53 | 1.88E−06 | 1 | −0.00611 | −0.00031 |
| CSNK2A2 | 2.32E−06 | 1 | −0.00601 | −0.00039 |
| OSBPL1A | 1.94E−06 | 1 | −0.00591 | −0.00033 |
| EIF2S1 | 1.95E−06 | 1 | −0.0055 | −0.00035 |
| CSDE1 | 2.05E−06 | 1 | −0.0054 | −0.00038 |
| PPP2R5D | 1.82E−06 | 1 | −0.00355 | −0.00051 |

### Discussion

In this report, the effect of age on dendritic spine density was evaluated within the precuneus in a large cohort of adult subjects spanning >70 years of normal aging. A significant negative correlation between the density of large dendritic spines and chronological age was identified. In this same cohort, precuneus tissue was then fractionated into homogenate and synaptosome fractions and detected a significant effect of age on 1839 of 5032 proteins detected in cellular homogenate and 914 of 4754 proteins detected in the synaptosome fraction. In an effort to nominate homogenate proteins that may lead to age-dependent large spine loss in the precuneus, a statistical mediation analysis was applied. 203 proteins was found which significantly mediated the effect of age on large dendritic spine density.

This report identifies age-related changes in the density of large dendritic spines within the precuneus. The loss of dendritic spines with increasing age (in the absence of neurodegenerative disease) has previously been reported in multiple brain areas, including DLPFC, hippocampus, cerebellum and even in subcortical areas (reviewed in [7]). Many of these studies opted to classify dendritic spines based on morphologic characteristics into predefined stubby, mushroom, thin, and filopodia categories and the absence of this classification scheme in this report may at first glance appear a limitation of the study.

However, this classification scheme was informed by the observation within this dataset that the correlation between dendritic spine density and age strengthened as dendritic spine size increases, such that the density of large, but not small or medium sized dendritic spines negatively correlated with age. Furthermore, there is increasing evidence that dendritic spines exist within a continuum of shapes rather than belonging to distinct morphologic classes[18] and that spine head volume is in fact the metric which correlates both with temporal spine stability and with spine functional metrics such as current flow through glutamatergic AMPA receptors[19], postsynaptic density size (reviewed in [20]), and smooth endoplasmic reticulum content[21]. Given these known structure-function relationships, the findings may suggest that the dendritic spines which demonstrate greater temporal stability and more potent signal transmission are lost with age.

Regarding the finding that the density of large dendritic spines declines with age and its possible implications for normal age-related cognitive decline, there are at least two possibilities. The first, and perhaps most intuitive, is that the degree of loss of large dendritic spines within the precuneus is linearly related to the degree of age-related cognitive deficits, an interpretation which is aligned with nonhuman primate studies which have demonstrated that synapse density correlates with performance on cognitive measures which decline during normal aging [23]. However, the correlation between age and the density of large dendritic spines was present across the entire cohort, and not driven solely by older subjects. An alternative possibility is that there exists a threshold of dendritic spine density below which detectable cognitive deficits ensue and above which cognitive function remains intact as assessed by currently available cognitive tests.

Relative preservation of dendritic spine density in the cerebral cortex, generally, and in the precuneus, specifically, during aging is anticipated to protect against the symptomatic manifestations of dementia (cognitive and functional decline) despite the presence of accumulating dementia-related neuropathologies (e.g. plaques, tangles) [5, 6, 35]. As such, the 203 proteins identified as statistical mediators of the effect of age on dendritic spine loss are potential targets for enhancing resilience to dementia onset by preventing or reversing age-associated dendritic spine loss. Any such effects may also have the added value of preventing or reversing normal age-associated cognitive decline.

In summary, the density of large dendritic spines within the precuneus negatively correlated with age. The most pronounced effects of age on the precuneus proteome were observed in the homogenate fraction with over 1800 proteins in homogenate being significantly correlated with age. Using statistical mediation analysis, 203 proteins mediating the effect of age on large dendritic spine loss were identified. These 203 proteins are thus potential druggable targets for preventing and reversing age-associated dendritic spine loss, with the subsequent goals of preventing or treating age-associated cognitive decline and protecting against dementia onset.

### REFERENCES

[1]. Penzes P, Cahill M E, Jones K A, VanLeeuwen J E, and Woolfrey K M, Dendritic spine pathology in neuropsychiatric disorders. Nat Neurosci, 2011. 14(3): p. 285-93.
[2]. Herms J and Dorostkar M M, Dendritic Spine Pathology in Neurodegenerative Diseases. Annu Rev Pathol, 2016. 11: p. 221-50.
[3]. Ji Y, Gong Y, Gan W, Beach T, Holtzman D M, and Wisniewski T, Apolipoprotein E isoform-specific regulation of dendritic spine morphology in apolipoprotein E transgenic mice and Alzheimer's disease patients. Neuroscience, 2003. 122(2): p. 305-15.
[4]. DeKosky S T and Scheff S W, Synapse loss in frontal cortex biopsies in Alzheimer's disease: correlation with cognitive severity. Ann Neurol, 1990. 27(5): p. 457-64.
[5]. Boros B D, Greathouse K M, Gearing M, and Herskowitz JH, Dendritic spine remodeling accompanies Alzheimer's disease pathology and genetic susceptibility in cognitively normal aging. Neurobiol Aging, 2019. 73: p. 92-103.
[6]. Boros B D, Greathouse K M, Gentry E G, Curtis K A, Birchall E L, Gearing M, et al., Dendritic spines provide cognitive resilience against Alzheimer's disease. Ann Neurol, 2017. 82(4): p. 602-614.
[7]. Dickstein D L, Weaver C M, Luebke J I, and Hof P R, Dendritic spine changes associated with normal aging. Neuroscience, 2013. 251: p. 21-32.
[8]. Colangeli S, Boccia M, Verde P, Guariglia P, Bianchini F, and Piccardi L, Cognitive Reserve in Healthy Aging and Alzheimer's Disease: A Meta-Analysis of fMRI Studies. Am J Alzheimers Dis Other Demen, 2016. 31(5): p. 443-9.
[9]. Aghakhanyan G, Vergallo A, Gennaro M, Mazzarri S, Guidoccio F, Radicchi C, et al., The Precuneus—A Witness for Excessive Abeta Gathering in Alzheimer's Disease Pathology. Neurodegener Dis, 2018. 18(5-6): p. 302-309.
[10]. Sperling R A, Laviolette P S, O'Keefe K, O'Brien J, Rentz D M, Pihlajamaki M, et al., Amyloid deposition is associated with impaired default network function in older persons without dementia. Neuron, 2009. 63(2): p. 178-88.
[11]. Sheline Y I, Raichle M E, Snyder A Z, Morris J C, Head D, Wang S, et al., Amyloid plaques disrupt resting state default mode network connectivity in cognitively normal elderly. Biol Psychiatry, 2010. 67(6): p. 584-7.
[12]. MacDonald M L, Alhassan J, Newman J T, Richard M, Gu H, Kelly R M, et al., Selective Loss of Smaller Spines in Schizophrenia. Am J Psychiatry, 2017. 174(6): p. 586-594.
[13]. Otsu N, A Threshold Selection Method from Gray-Level Histograms. IEEE Transactions on Systems, Man, and Cybernetics, 1979. 9(1): p. 62-66.
[14]. McKinney B C, MacDonald M L, Newman J T, Shelton M A, DeGiosio R A, Kelly R M, et al., Density of small dendritic spines and microtubule-associated-protein-2 immunoreactivity in the primary auditory cortex of subjects with schizophrenia. Neuropsychopharmacology, 2019. 44(6): p. 1055-1061.
[15]. O'Donnell C, Nolan M F, and van Rossum M C, Dendritic spine dynamics regulate the long-term stability of synaptic plasticity. J Neurosci, 2011. 31(45): p. 16142-56.
[16]. Langfelder P and Horvath S, WGCNA: an R package for weighted correlation network analysis. BMC Bioinformatics, 2008. 9: p. 559.
[17]. Wang H, Azuaje F, Bodenreider O, and Dopazo J, Gene Expression Correlation and Gene Ontology-Based Similarity: An Assessment of Quantitative Relationships. Proc IEEE Symp Comput Intell Bioinforma Comput Biol, 2004. 2004: p. 25-31.
[18]. Pchitskaya E and Bezprozvanny I, Dendritic Spines Shape Analysis-Classification or Clusterization? Perspective. Front Synaptic Neurosci, 2020. 12: p. 31.
[19]. Matsuzaki M, Ellis-Davies G C, Nemoto T, Miyashita Y, Lino M, and Kasai H, Dendritic spine geometry is critical for AMPA receptor expression in hippocampal CA1 pyramidal neurons. Nat Neurosci, 2001. 4(11): p. 1086-92.
[20]. Hayashi Y and Majewska A K, Dendritic spine geometry: functional implication and regulation. Neuron, 2005. 46(4): p. 529-32.
[21]. Spacek J and Harris K M, Three-dimensional organization of smooth endoplasmic reticulum in hippocampal CA1 dendrites and dendritic spines of the immature and mature rat. J Neurosci, 1997. 17(1): p. 190-203.
[22]. Hao J, Rapp P R, Janssen W G, Lou W, Lasley B L, Hof P R, et al., Interactive effects of age and estrogen on cognition and pyramidal neurons in monkey prefrontal cortex. Proc Natl Acad Sci U S A, 2007. 104(27): p. 11465-70.
[23]. Dumitriu D, Hao J, Hara Y, Kaufmann J, Janssen W G, Lou W, et al., Selective changes in thin spine density and morphology in monkey prefrontal cortex correlate with aging-related cognitive impairment. J Neurosci, 2010. 30(22): p. 7507-15.
[24]. Villa K L, Berry K P, Subramanian J, Cha J W, Oh W C, Kwon H B, et al., Inhibitory Synapses Are Repeatedly Assembled and Removed at Persistent Sites In Vivo. Neuron, 2016. 89(4): p. 756-69.
[25]. Harris K M and Stevens J K, Dendritic spines of CA 1 pyramidal cells in the rat hippocampus: serial electron microscopy with reference to their biophysical characteristics. J Neurosci, 1989. 9(8): p. 2982-97.
[26]. Schikorski T and Stevens C F, Quantitative fine-structural analysis of olfactory cortical synapses. Proc Natl Acad Sci U S A, 1999. 96(7): p. 4107-12.
[27]. Mensch S, Baraban M, Almeida R, Czopka T, Ausborn J, El Manira A, et al., Synaptic vesicle release regulates myelin sheath number of individual oligodendrocytes in vivo. Nat Neurosci, 2015. 18(5): p. 628-30.
[28]. Hughes A N and Appel B, Oligodendrocytes express synaptic proteins that modulate myelin sheath formation. Nat Commun, 2019. 10(1): p. 4125.
[29]. Gibson E M, Purger D, Mount C W, Goldstein A K, Lin G L, Wood L S, et al., Neuronal activity promotes oligodendrogenesis and adaptive myelination in the mammalian brain. Science, 2014. 344(6183): p. 1252304.
[30]. Pease-Raissi S E and Chan J R, Building a (w)rapport between neurons and oligodendroglia: Reciprocal interactions underlying adaptive myelination. Neuron, 2021. 109(8): p. 1258-1273.
[31]. Mount C W and Monje M, Wrapped to Adapt: Experience-Dependent Myelination. Neuron, 2017. 95(4): p. 743-756.
[32]. McKenzie I A, Ohayon D, Li H, de Faria J P, Emery B, Tohyama K, et al., Motor skill learning requires active central myelination. Science, 2014. 346(6207): p. 318-22.
[33]. Etnier J L and Landers D M, Motor performance and motor learning as a function of age and fitness. Res Q Exerc Sport, 1998. 69(2): p. 136-46.
[34]. Voelcker-Rehage C, Motor-skill learning in older adults—a review of studies on age-related differences. Eur Rev Aging Phys Act, 2008. 5: p. 5-16.
[35]Mi Z, Abrahamson E E, Ryu A Y, Fish K N, Sweet R A, Mufson E J, Ikonomovic M D. Loss of precuneus dendritic spines immunopositive for spinophilin is related to cognitive impairment in early Alzheimer's disease. Neurobiol Aging. 2017; 55:159-166.

### Example 2

### Method to Identify Drug Targets

In Example 1 203 protein mediators of age-related dendritic spine loss were identified. In this example computational systems pharmacology were used to identify existing drugs that are known to: 1) act on one or more of the 203 proteins in the direction predicted to reverse dendritic spine loss (e.g. if increased protein levels were associated with reduced dendritic spines, drugs that inhibit the protein target were identified); 2) have a net effect on the 203 proteins that opposes (i.e. is predicted to reverse) the effects of age on dendritic spines.

Material: 203 mediator proteins identified in Example 1. Drug targets and action data obtained from Drugbank.

Identify related drugs: If a drug has a known target(s) that are included in the 203 proteins, it is considered as a related drug. All targets of these drugs and their action are extracted from the database.

Action alignment: Since increase dendritic spine density is wanted, the drug should be able to activate the proteins that can up-regulate the dendritic spine density (Table 3B,3D) or inhibit the proteins that can down-regulate the dendritic spine density (Table 3C,3E). The drug-target pairs are filtered by the desired action resulting in 15 drug-target pairs for 11 drugs.

Quality check: For the 11 drugs identified in the previous step, their actions at all of their targets were looked at to make sure no competing actions are presented by a certain drug. One drug (fostamatinib) was eliminated because of such competing actions.

Validation with post-treatment gene signature where available: Data were extracted from LINC1000 database for all drugs about which information was available from testing in human neuronal cells, with a drug exposure of at least 24 hours. Six drugs had available LINC1000 data meeting the above criteria: fostamatinib, metformin, leflunomide, bosutinib, dabrafenib, and pazopanib.

Signed Jaccard Score [1] is used to quantitatively evaluate whether the net (direct and indirect) effects of each drug at all 203 mediator proteins opposes (i.e. is predicted to reverse) the effects of age on dendritic spines. Computationally identified drugs that reverse the protein mediator signature of age-dependent spine loss and their gene targets within the list of mediator proteins are shown in Table 4. Though each was identified based on known effects on the genes listed, each is predicted to have an indirect effect on multiple proteins among the 203 mediator proteins so as to yield a net predicted reversal of the effects of age on dendritic spines.

TABLE 4
Computationally identified drugs that reverse the protein mediator
signature of age-dependent spine loss and their gene target
within the list of mediator proteins in Tables 3A-3E.

| Drug | Gene Target |
|---|---|
| Metformin | GPD1 |
| Pentosan polysulfate | FGF1 |
| Amlexanox | FGF1 |
| Leflunomide | PTK2B |
| Prasterone | GRIN1 |
| Glutathione disulfide | MGST1 |
| Pazopanib | FGF1 |
| Bosutinib | CAMK2G |
| Dabrafenib | LIMK1 |
| Baricitinib | PTK2B |

### REFERENCE

1. Qi, X.; Shen, M.; Fan, P.; Guo, X.; Wang, T.; Feng, N.; Zhang, M.; Sweet, R. A.; Kirisci, L.; Wang, L., The Performance of Gene Expression Signature-Guided Drug-Disease Association in Different Categories of Drugs and Diseases. Molecules 2020, 25 (12), 2776.

### Example 3

### Amlexanox Treatment Alters Brain Proteins in the Directions Which are Predicted by Our Computational Model to Prevent or Reverse Age-Associated Dendritic Spine Loss

As proof of principle, our prediction for one of the drugs identified in Example 2, amlexanox was empirically evaluated.

### Materials and Methods

Drug administration and animal studies. The initial dose of amlexanox (100 mg/kg), derived from a previously published study in mice demonstrating CNS effects of IP amlexanox treatment [1] was tested in a separate cohort of 6 C57B16/J (WT) mice for comparison with equivalent volume drug vehicle (50% DMSO in normal saline). A modified tolerability score [2] was used to estimate drug tolerability. Based on elevated tolerability scores, the dose was twice decreased by half for a final dose of 25 mg/kg. Male and female 15-month-old WT mice were randomly allocated to receive either 25 mg/kg amlexanox (n=9) or equivalent volume vehicle (n=10) for 5 days. Treatment groups were balanced for sex. On day 5, mice were sacrificed, perfused with normal saline, and the right cerebral cortex was isolated and immediately frozen on dry ice for proteomics.

Sample Preparation. Tissue homogenate was obtained using a Bead Mill Fast Prep-24 (MP Biomedicals). Tissue was transferred into a Lysing Matrix D tube with 750 μl of homogenization solution (5% SDS with MS-SAFE Protease and Phosphatase Inhibitor) and homogenized at 6 m/s for 40 s. Homogenate was centrifuged at 13.2×g for 10 min, the supernatant was transferred to a microcentrifuge tube and total protein was quantified with BCA.

10 μg of protein from each subject was digested with trypsin at a ratio of 1:20 using S-trap micro columns (Protifi) and desalted using Pierce Peptide Desalting Spin Columns (ThermoFisher Scientific). Samples were resuspended in a solution of 3% acetonitrile/0.1% formic acid in water at a concentration of 0.5 μg/μl. A pooled control (PC) was created by mixing 1 μg of peptides (2 μL) from each sample.

Mass Spectrometry. Mass spectrometry analysis was conducted on a Thermo Fisher QE-HFX coupled to an Easy-nLC 1200. Approximately 1 μg of each sample was loaded onto an EASY-Spray PepMap RSLC C18 column (2 μm, 100 A, 75 μm×50 cm) and eluted at 300 nl/min over a 60-minute gradient. An instrument blank and the PC were injected after every 9th sample injection to evaluate the instrument variability over the course of the experiment. MS1 spectra were collected at 60,000 resolution with a full scan range of 350-1400 m/z, a maximum injection time of 50 ms and the automatic gain control (AGC) set to 3e6. The precursor selection window was 1.4 m/z and fragmentation was carried out with HCD at 28% NCE. MS2 were collected with a resolution of 30,000, a maximum injection time of 50 ms and the AGC set to 1e5 and the dynamic exclusion time set to 90 s.

Peptide Data Analysis. Raw data files were analyzed with Proteome Discoverer (ThermoFisher Scientific) and searched against the mouse SwissProt database using Sequest HT (missed cleavages=2; minimum peptide length=6; precursor mass tolerance=10 ppm; dynamic modifications: Oxidation/+15.995 Da, Acetyl/+42.011 Da, Met-loss/−131.040 Da, Met-loss+Acetyl/-83.030 Da; static modifications: Carbamidomethyl/+57.021 Da). Peptide spectral matches were filtered using Percolator with a dCN=0.05 and 1-DR=1%.

Statistical Analysis. Protein-level data were extracted from Proteome Discoverer. Proteins with a combined Q-value=0.01 or greater, not quantified in 100% of samples or with CV>0.3 in pooled controls were filtered out. Protein-level estimates were transformed on a log2 scale and two-sided t-tests were used to calculate unadjusted p values for comparison between treatment groups.

### Results

2860 proteins were quantified in all mice. The relative abundance (Amlexanox/Control) of each protein is shown in FIG. 7 on a log2 scale. Proteins with unadjusted p-values <0.05 from two-sided t-tests between amlexanox and control groups are annotated. The most significantly altered protein after amlexanox treatment was Fibroblast Growth Factor Receptor 2 (FGFR2, amlexanox/control=0.69, unadjusted p value of log2 fold change=0.0012). FGFR2, the most highly expressed fibroblast growth factor receptor in cerebral cortex, is a receptor for Fibroblast growth Factor 1, the protein target of amlexanox. Other Fibroblast Growth Factor Receptors were not quantified in our assay. This finding strongly suggests that the target pathway (reduction of FGF1 signaling) was engaged in the brain by amlexanox treatment.

then the net effect of amlexanox treatment was evaluated on proteins which, in our human data, were associated with age related dendritic spine density reductions. The Signed Jaccard Index (SJI) [3] was calculated to estimate the net overlap of amlexanox-induced protein perturbations in mice with the effects of proteins on dendritic spines in humans. The relative log2-transformed abundances for each protein in amlexanox/control groups were compared with the effects of each protein on the density of large dendritic spines extracted from the mediation analysis from our human cohort (described in Example 1) as follows:

$$SJI = \frac{( \begin{matrix}\frac{↑ both}{uniquely ↑ both} + \frac{↓ both}{uniquely ↓ both} - \\ \frac{↑ human and ↓ mouse}{uniquely ↑ human or ↓ mouse} - \frac{↓ human and ↑ mouse}{uniquely ↓ human or ↑ mouse}\end{matrix} )}{2}$$

$$SJI = \frac{( 0.205 + 0.206 - 0.184 - 0.201 )}{2} = 0.013$$

In this instance, the SJI, as desired, was positive, indicating that amlexanox shifted the mouse cerebral cortex proteome in a direction associated with increased spine densities in human. An SJI of 0.013 compares favorably to the mean SJI reported for all drug-disease indication pairs (absolute value=0.004), indicating it is compatible with therapeutic activity. However, the SJI of 0.013 observed was lower in magnitude than the mean SJI reported for drug-disease pairs indicated specifically for CNS disorders (absolute value=0.033) [3].

### REFERENCES

1. Liu Y, Lu J, Zhang Z, Zhu L, Dong S, Guo G, et al., Amlexanox, a selective inhibitor of IKBKE, generates anti-tumoral effects by disrupting the Hippo pathway in human glioblastoma cell lines. Cell Death Dis, 2017. 8(8): p. e3022.
2. Aston W J, Hope D E, Nowak A K, Robinson B W, Lake R A, and Lesterhuis W J, A systematic investigation of the maximum tolerated dose of cytotoxic chemotherapy with and without supportive care in mice. BMC Cancer, 2017. 17(1): p. 684.
3. Qi X, Shen M, Fan P, Guo X, Wang T, Feng N, et al., The Performance of Gene Expression Signature-Guided Drug-Disease Association in Different Categories of Drugs and Diseases. Molecules, 2020. 25(12).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method of treating, preventing, or reducing age-associated dendritic spine density loss in a subject, the method comprising administering a compound selected from:
metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof.

2. The method of claim 1, wherein the compound is selected from metformin, pentosan polysulfate, amlexanox, leflunomide, prasterone, glutathione disulfide, pazopanib, bosutinib, dabrafenib, or baricitinib, or a pharmaceutically acceptable salt, prodrug, or derivative thereof.

3. The method of claim 1, wherein the compound is metformin, or a pharmaceutically acceptable salt, prodrug, or derivative thereof.

4. The method of claim 1, wherein the compound is pentosan polysulfate, or a pharmaceutically acceptable salt, prodrug, or derivative thereof.

5. The method of claim 1, wherein the compound is amlexanox, or a pharmaceutically acceptable salt, prodrug, or derivative thereof.

6. The method of claims 1, wherein the compound is leflunomide, or a pharmaceutically acceptable salt, prodrug, or derivative thereof.

7. The method of claim 1, wherein the compound is prasterone, or a pharmaceutically acceptable salt, prodrug, or derivative thereof.

8. The method of claim 1, wherein the compound is glutathione disulfide, or a pharmaceutically acceptable salt, prodrug, or derivative thereof.

9. The method of claim 1, wherein the compound is pazopanib, or a pharmaceutically acceptable salt, prodrug, or derivative thereof.

10. The method of claim 1, wherein the compound is bosutinib, or a pharmaceutically acceptable salt, prodrug, or derivative thereof.

11. The method of claim 1, wherein the compound is dabrafenib, or a pharmaceutically acceptable salt, prodrug, or derivative thereof.

12. The method of claim 1, wherein the compound is baricitinib, or a pharmaceutically acceptable salt, prodrug, or derivative thereof.

13. The method of claim 1, wherein the subject has dementia.

14. The method of claim 1, wherein the method protects against neurodegenerative dementia onset.

15. A method of increasing the activity, level, or any combination thereof of a mediator protein selected from the list of mediator protein in Tables 3B and 3D in a subject, the method comprising administering a compound selected from: metformin, pentosan polysulfate, amlexanox, leflunomide, prasterone, glutathione disulfide, pazopanib, bosutinib, dabrafenib, baricitinib or a pharmaceutically acceptable salt, prodrug, or derivative thereof; or any combination thereof.

16. The method of claim 15, wherein the compound is selected from: glutathione disulfide, prasterone, or a pharmaceutically acceptable salt, prodrug, or derivative thereof; or any combination thereof.

17. The method of claim 15, wherein the compound is glutathione disulfide, or a pharmaceutically acceptable salt, prodrug, or derivative thereof.

18. The method of claim 15, wherein the subject has age-associated dendritic spine density loss, age related cognitive decline, or a combination thereof.

19. A method of decreasing the activity, level, or any combination of a mediator protein selected from the list of mediator protein in Tables 3C and 3E in a subject, the method comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof.

20. A method of enhancing cognitive resilience in a subject comprising administering a compound selected from: metformin, pentosan polysulfate, amlexanox, leflunomide, prasterone, glutathione disulfide, pazopanib, bosutinib, dabrafenib, baricitinib, or a pharmaceutically acceptable salt, prodrug, or derivative thereof; or any combination thereof.
